(19) United States
(12) Patent Application Publication
(10) Pub. No.: US 2022/0148678 A1
(43) Pub. Date: May 12, 2022

(54) METHODS FOR DETERMINING A DESIGNABLE REGION OF OLIGONUCLEOTIDES

(71) Applicant: SEEGENE, INC., Seoul (KR)

(72) Inventors: Ju-Hee BAE, Seoul (KR); Je-Hwan PARK, Seoul (KR)

(21) Appl. No.: 17/434,455

(22) PCT Filed: Feb. 28, 2020
(86) PCT No.: PCT/KR2020/002921

§ 371 Date: Aug. 27, 2021

(30) Foreign Application Priority Data
Feb. 28, 2019 (KR) 10-2019-0024076

(51) Int. Cl. G16B 30/10; G16B 25/10

(52) U.S. Cl. CPC G16B 30/10; G16B 25/10

(57) ABSTRACT

The present invention relates to technologies for determining a designable region of oligonucleotides in a plurality of target nucleic acid sequences having sequence similarity. In determining a conservative region in a plurality of nucleic acid sequences, unlike the conventional methods which is an empirical and manually selected methods, the present invention provides a more logical and efficient method by adopting a strategy of generating oligonucleotide sticks having sequence information about non-conservative positions within a predetermined allowable number or a minimum number of sequence patterns within a predetermined allowable number of sequence patterns while having a predetermined length or more from the alignment results of a plurality of target nucleic acid sequences and has excellent speed and accuracy.

### CROSS-REFERENCE TO RELATED APPLICATIONS

This application claims priority from Korean Patent Application No. 2019-0024076, filed on Feb. 28, 2019 in the Korean Intellectual Property Office, the disclosure of which is incorporated herein by reference.

### BACKGROUND OF THE INVENTION

### Field of the Invention

The present invention relates to technologies for determining a designable region of oligonucleotides in a plurality of target nucleic acid sequences having sequence similarity.

### Description of the Related Art

A variety of techniques have been developed to detect target nucleic acid molecules of pathogens and identify these target nucleic acid molecules, and these are collectively referred to as molecular diagnostics. Most of the molecular diagnostic techniques use oligonucleotides such as primers and probes hybridizable with target nucleic acid molecules.

To date, there have been many advances made in molecular diagnostic technologies. However, there are still technical challenges to be solved in the diagnosis of pathogens having genomes that exhibit genetic diversity or genetic variability.

Genetic diversity or genetic variability has been reported in various genomes. In particular, genetic diversity is most frequently found and occurs in viral genomes (Bastien N. et al., Journal of Clinical Microbiology, 42:3532(2004); Peret T C. et al., Journal of Infectious Diseases, 185:1660(2002); Ebihara T. et al., Journal of Clinical Microbiology, 42:126(2004); Jenny-Avital E R. et al. Clinical Infectious Diseases, 32:1227(2001); Duffy S. et. al., Nat. Rev. Genet. 9(4):267-76(2008); Tong Y G et. al., Nature. 22:526(2015)).

In detecting a pathogen with genetic diversity, designing oligonucleotides while taking into account a certain sequence of a target nucleic acid molecule of this pathogen is very likely to lead to false negative results. Thus, in order to determine whether a certain pathogen is present in an unknown sample, probes or primers should be designed in consideration of all nucleic acid sequences or as many nucleic acid sequences as possible of known genetic diversity for one target nucleic acid molecule of this certain pathogen. In order to detect a target nucleic acid molecule exhibiting such genetic diversity, two approaches have been largely developed.

The first method is to design a degenerate oligonucleotide. Typically, a region including sequences having sequence similarity is found in the alignment of all the nucleic acid sequences of a certain gene having genetic diversity, and the certain gene is detected with a desired coverage using a degenerate primer or probe (including a degenerate base at a variation site) that is hybridized with the region. When a certain gene is not detected with a desired coverage using the degenerate oligonucleotide, the second method below is used.

The second method detects a target nucleic acid molecule using a plurality of oligonucleotides that are hybridized with a plurality of nucleic acid sequences of a target nucleic acid molecule exhibiting genetic diversity. For example, when targeting M gene of influenza A virus, all nucleic acid sequences known of the M gene are aligned and probes are designed capable of covering all of these nucleic acid sequences. In such a case, a plurality of probes (probes with different probing positions each other) are designed since a single probe cannot cover all M genes of various sequences. In such a case, a degenerate base may also be introduced into the plurality of probes to further extend coverage.

In order to select any one of the two methods, it is most important to determine a region in target nucleic acid sequences for being capable of designing an oligonucleotide so that nucleic acid sequences with diversity of a target nucleic acid molecule can be detected using the oligonucleotide or a combination thereof. Considering convenience, efficiency, and economy of an analysis, it is desirable to select a designable region of an oligonucleotide in a plurality of target nucleic acid sequences having sequence similarity and design from the designable region an oligonucleotide to be used in any one of the two methods.

Conventionally, in order to detect nucleic acid sequences with diversity of a target nucleic acid molecule, researchers have selected a conservative region excluding a position at which a variation base exists while observing alignment results of a plurality of target nucleic acid sequences by the naked eye and then designed an oligonucleotide covering the plurality of target nucleic acid sequences from the region, and for target coverage extension, determined introduction positions and the number of degenerate bases to be introduced into the designed oligonucleotide, or an optimal combination of oligonucleotides.

However, in selecting a conservative region for designing oligonucleotides that cover the maximum sequences when the number of target nucleic acid sequences is large, the conventional methods had a problem that it not only take a long time but also show poor accuracy. Moreover, when a plurality of target nucleic acid sequences have large genetic diversity, the conventional methods had a drawback showing poor accuracy and economy since a designable region of an oligonucleotide fails to be selected, a large number of degenerate bases are introduced into one oligonucleotide, or a plurality of oligonucleotides are combined, and had a problem in that even it is not possible to select a region for designing oligonucleotides, so it takes time to select a different target nucleic acid molecule other than the desired target nucleic acid molecule.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive researches to develop a method being capable of providing a region in target nucleic acid sequences which is capable of efficiently designing an oligonucleotide (e.g., a primer and a probe) used in amplifying and detecting a target nucleic acid molecule, especially a target nucleic acid molecule with genetic diversity. As a result, the present inventors have found that in order to design an oligonucleotide used to amplify and detect a target nucleic acid molecule, oligonucleotide sticks having sequence information about the number of non-conservative positions or the number of sequence patterns can be generated from an alignment of a plurality of target nucleic acid sequences and the oligonucleotide sticks can be used to provide, with speed and accuracy, a designable region being able to cover the plurality of target nucleic acid sequences by using one oligonucleotide group (e.g., one primer pair and/or one probe) or a plurality of oligonucleotide groups (e.g., two or more primer pairs and/or two or more probes).

Accordingly, it is an object of this invention to provide a method for determining a designable region of oligonucleotides in a plurality of target nucleic acid sequences having sequence similarity.

It is another object of this invention to provide a computer readable storage medium containing instructions to configure a processor to perform a method for determining a designable region of oligonucleotides in a plurality of target nucleic acid sequences having sequence similarity.

Other objects and advantages of the present invention will become apparent from the detailed description to follow taken in conjugation with the appended claims and drawings.

### BRIEF DESCRIPTION OF THE DRAWINGS

FIG. 1 is a flow diagram showing a method for determining a designable region of oligonucleotides by generating single sticks according to an embodiment of the present invention.

FIG. 2 shows a procedure of generating single sticks according to an embodiment of the present invention. In alignment positions of a plurality of target nucleic acid sequences, V represents a variation position (a non-conservative position) and G represents a gap-containing position.

FIG. 3 represents a procedure of selecting the portion satisfying a predetermined GC content in the generated single stick according to an embodiment of the present invention.

FIG. 4 shows a procedure of selecting single sticks, which have generated and passed through a basic filter, using an amplicon filter (amplicon region forming ability), according to an embodiment of the present invention.

FIG. 5 is a flow diagram showing a method for determining a designable region of oligonucleotides by generating pattern sticks according to an embodiment of the present invention.

FIG. 6 shows a procedure of generating pattern sticks according to an embodiment of the present invention. In alignment positions of a plurality of target nucleic acid sequences, P represents a sequence pattern change position and G represents a gap-containing position.

FIG. 7 shows the results of determining a designable region of oligonucleotides by generating single sticks on the alignment of a plurality of hemagglutinin-neuraminidase (HN) gene sequences of Human parainfluenza virus type 2 (PIV2) according to an embodiment of the present invention. In the determined designable region (DR), a portion indicated by A+B represents a designable region determined according to an example of the present invention and a previously known design region which is manually selected by the naked eye, and each of the other portions indicated by A represents a designable region determined according to an example of the present invention.

FIG. 8 shows the results of determining a designable region of oligonucleotides by generating pattern sticks on the alignment of a plurality of F gene sequences of Human metapueumovirus (hPMV) according to an embodiment of the present invention. In the determined designable region (DR), a portion indicated by A+B represents a designable region determined according to an example of the present invention and a previously known design region which is manually selected by the naked eye and each of the other portions indicated by A represents a designable region determined according to an example of the present invention.

### DETAILED DESCRIPTION OF THIS INVENTION

### I. Method for Determining Designable Region of Oligonucleotide (the First Aspect)

In one aspect of the present invention, there is provided a method for determining a designable region of oligonucleotides in a plurality of target nucleic acid sequences having sequence similarity, comprising:

(a) selecting a start position from alignment positions of a plurality of target nucleic acid sequences; wherein the alignment positions comprise a conservative position and a non-conservative position of nucleotides of the plurality of target nucleic acid sequences that are aligned, the conservative position has one type of bases exhibiting conservativity, and the non-conservative position has two or more types of bases exhibiting non-conservativity;

(b) selecting as an end position a position comprising a non-conservative position within a predetermined allowable number from the start position;

(c) generating an oligonucleotide stick composed of a region from the start position to the end position; wherein the oligonucleotide stick comprises sequence information determined by a plurality of target nucleic acid sequences that are aligned in the region;

(d) repeating the generation of an oligonucleotide stick by selecting at least one start position different from the start position in step (a); and

(e) determining as a designable region of oligonucleotides regions in an alignment of the plurality of target nucleic acid sequences, which correspond to the regions of the oligonucleotide sticks.

A first aspect of the present invention relates to a method in which oligonucleotide sticks having sequence information about the number of non-conservative positions are generated from alignment positions of a plurality target nucleic acid sequences, and then a designable region of oligonucleotides is determined on the basis of the oligonucleotide sticks. The method according to the first aspect of the present invention is referred to as a single stick manner, and as used in the method according to the first aspect of the present invention, the terms “oligonucleotide stick” and “single stick” may be exchangeably used with each other.

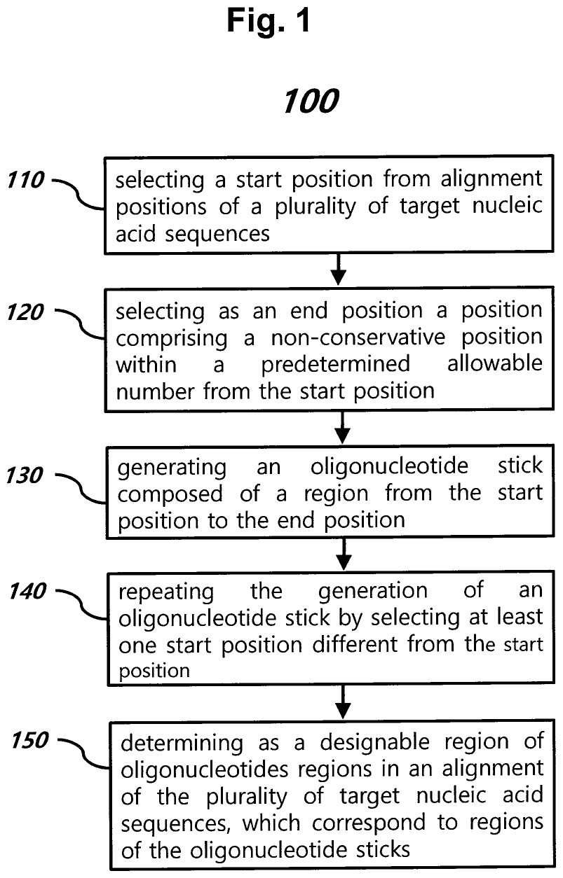

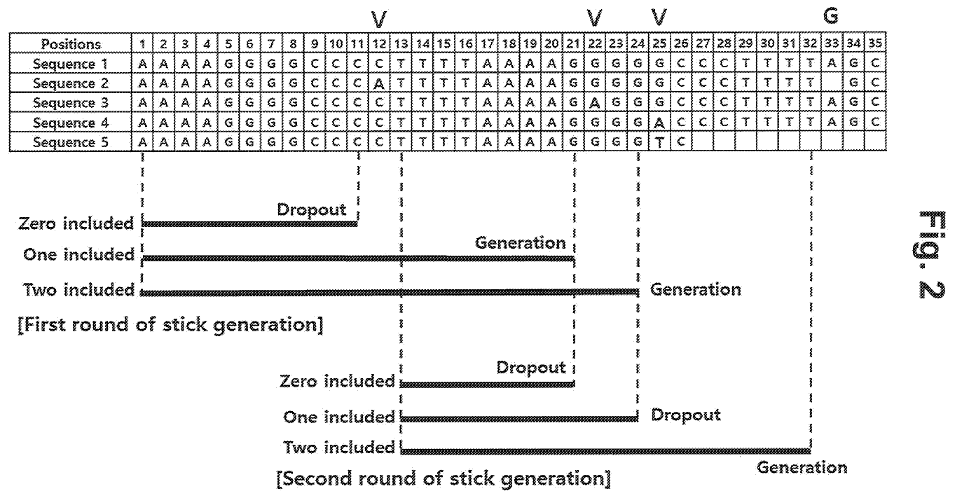

FIG. 1 is a flow diagram of steps for implementing a first aspect of the present invention according to an embodiment of the present invention, and FIG. 2 shows the generation process of an oligonucleotide stick during the implementation of the first aspect of the present invention according to an embodiment of the present invention. A method according to the first aspect of the present invention will be described with reference to FIG. 1 and FIG. 2 as below:

Step (a): Selecting Start Position from Alignment Positions of Plurality of Target Nucleic Acid Sequences (110)

First, a start position is selected from alignment positions of a plurality of target nucleic acid sequences. The alignment positions comprise a conservative position and a non-conservative position of nucleotides of the plurality of target nucleic acid sequences that are aligned, the conservative position has one type of bases exhibiting conservativity, and the non-conservative position has two or more types of bases exhibiting non-conservativity.

The term used herein “target nucleic acid molecule”, “target molecule” or “target nucleic acid” refers to a nucleotide molecule in an organism intended to detect. Generally, the target nucleic acid molecule has a certain name and includes an entire genome and all nucleotide molecules that make up a genome (e.g., gene, pseudogene, non-coding sequence molecule, untranslated region and some regions of genome).

The target nucleic acid molecule includes, for example, prokaryotic cell (e.g., Mycoplasma pneumoniae, Chlamydophila pneumoniae, Legionella pneumophila, Haemophilus influenzae, Streptococcus pneumoniae, Bordetella pertussis, Bordetella parapertussis, Neisseria meningitidis, Listeria monocytogenes, Streptococcus agalactiae, Campylobacter, Clostridium difficile, Clostridium perfringens, Salmonella, Escherichia coli, Shigella, Vibrio, Yersinia enterocolitica, Aeromonas, Chlamydia trachomatis, Neisseria gonorrhoeae, Trichomonas vaginalis, Mycoplasma hominis, Mycoplasma genitalium, Ureaplasma urealyticum, Ureaplasma parvum, Mycobacterium tuberculosis) nucleic acid, eukaryotic cell (e.g., protozoan and parasitic animal, fungus, yeast, higher plant, lower animal, and higher animal including mammal and human) nucleic acid, virus nucleic acid or viroid nucleic acid. Parasite of the eukaryotic cell includes, for example, Giardia lamblia, Entamoeba histolytica, Cryptosporidium, Blastocystis hominis, Dientamoeba fragilis, and Cyclosporacayetanensis. Example of such virus includes influenza A virus (Flu A), influenza B virus (Flu B), respiratory syncytial virus A (RSV A), respiratory syncytial virus B (RSVB), parainfluenza virus 1 (PIV 1), parainfluenza virus 2 (PIV 2), parainfluenza virus 3 (PIV 3), parainfluenza virus 4 (PIV 4), metapneumovirus (MPV), human enterovirus (HEV), human bocavirus (HBoV), human rhinovirus (HRV), coronavirus and adenovirus, which cause respiratory diseases; norovirus, rotavirus, adenovirus, astrovirus, and sapovirus, which cause gastrointestinal disorders. The virus also includes, for example, human papillomavirus (HPV), middle east respiratory syndrome-related coronavirus (MERS-CoV), dengue virus, herpes simplex virus (HSV), human herpes virus (HHV), epstein-barr virus (EMV), varicella zoster virus (VZV), cytomegalovirus (CMV), HIV, hepatitis virus, and poliovirus.

The term used herein “target nucleic acid sequence” or “target sequence” is to represent a target nucleic acid molecule as a certain sequence.

One target nucleic acid molecule, for example, one target gene, may have a certain target nucleic acid sequence; otherwise for a target nucleic acid molecule exhibiting genetic diversity or genetic variability, it may have a plurality of target nucleic acid sequences with diversity.

The plurality of target nucleic acid sequences in the present invention are target nucleic acid sequences having sequence similarity. Specifically, the target nucleic acid sequences having sequence similarity may be a plurality of target nucleic acid sequences of one target nucleic acid molecule or a plurality of target nucleic acid sequences of two or more target nucleic acid molecules.

According to an embodiment, the plurality of target nucleic acid sequences in the present invention are a plurality of nucleic acid sequences having sequence similarity for one target nucleic acid molecule exhibiting genetic diversity.

For example, the plurality of target nucleic acid sequences used in the present invention are a plurality of nucleic acid sequences having sequence similarity for a target nucleic acid molecule that exhibits genetic diversity, such as a viral genome sequence. For example, when influenza A virus is to be detected and the M gene is determined as a target nucleic acid molecule, target nucleic acid sequences with diversity of the M gene of the influenza A virus may be used. The full-length nucleic acid sequence as well as a partial sequence of the M gene of the influenza A virus may be used. The influenza A virus includes a variety of subtypes and variants, and their genomic sequences are different from each other. Therefore, when the influenza A virus is to be detected without a false negative result, a region in target nucleic acid sequences, which is for designing an oligonucleotide, should be determined considering various target nucleic acid sequences of a target nucleic acid molecule of the influenza A virus originated from such genetic diversity.

More particularly, the plurality of target nucleic acid sequences are a whole genome sequence, a partial sequence of a genome, or a plurality of nucleic acid sequences of one gene of virus or bacteria having genetic diversity.

According to an embodiment of the present invention, the plurality of target nucleic acid sequences are a plurality of nucleic acid sequences corresponding to homologues of a plurality of organisms, having the same function, the same structure, or the same gene name. The organisms mean organisms belonging to one genus, species, subspecies, subtype, genotype, serotype, strain, isolate or cultivar. The homologues include proteins and nucleic acid molecules. In this embodiment, a plurality of nucleic acid sequences of homologous biomolecules (e.g., protein or nucleic acid) of a plurality of organisms, having the same function (e.g., a biological function of a protein encoded by a nucleic acid sequence), the same structure (e.g., a tertiary structure of a protein encoded by a nucleic acid sequence) or the same gene name, are used. For example, a plurality of nucleic acid sequences known for the E5 gene of HPV type 16 may be considered as nucleic acid sequences of isolates of HPV type 16.

According to an embodiment, the target nucleic acid sequence includes nucleic acid sequences belonging to a subclass of any biological classification (e.g., genus, species, subtype, genotype, serotype and subspecies). For example, when the target nucleic acid sequence is HPV type 16, the target nucleic acid sequence may include nucleic acid sequences belonging to a subclass thereof.

According to an embodiment of the present invention, the plurality of target nucleic acid sequences are at least 3, at least 5, at least 10, at least 20, at least 30, at least 40, at least 50, at least 60, at least 70, at least 80, at least 90, at least 100, at least 200, at least 300, or at least 500 nucleic acid sequences. For example, a plurality of target nucleic acid sequences are sequences 1 to 5 in FIG. 2.

A plurality of target nucleic acid sequences may be provided using various sequence databases. For example, a plurality of desired target nucleic acid sequences may be collected and provided from a publicly accessible database, such as GenBank, European Molecular Biology Laboratory (EMBL) sequence database, and DNA DataBank of Japan (DDBJ).

In the present invention, alignment of target nucleic acid sequences may be performed according to various methods (e.g., global alignment and local alignment) and algorithms known in the art.

Various methods and algorithms for alignment are described in Smith and Waterman, Adv. Appl. Math. 2:482(1981); Needleman and Wunsch, J. Mol. Bio. 48:443(1970), Pearson and Lipman, Methods in Mol. Biol. 24: 307-31(1988); Higgins and Sharp, Gene 73:237-44(1988); Higgins and Sharp, CABIOS 5:151-3(1989); Corpet et al., Nuc. Acids Res. 16: 10881-90 (1988); Huang et al., Comp. Appl. BioSci. 8:155-65(1992) and Pearson et al., Meth. Mol. Biol. 24:307-31(1994). The NCBI Basic Local Alignment Search Tool (BLAST) (Altschul et al., J. Mol. Biol. 215:403-10(1990)) is accessible from NCBI (National Center for Biological Information) and may be used in conjunction with sequence analysis programs such as blastn, blasm, blastx, tblastn and tblastx on the Internet. BLSAT is available at http://www.ncbi.nlm.nih.gov/BLAST/. A comparison of sequence similarity using this program may be found at http://www.ncbi.nlm.nih.gov/BLAST/blast help.html.

According to the present invention, a plurality of target nucleic acid sequences are aligned, and a start position is selected from alignment positions.

The term used herein, “alignment positions” refers to positions at which nucleotides of a plurality of target nucleic acid sequences are aligned according to the homology of the plurality of target nucleic acid sequences, and the respective positions are expressed as serial numbers.

The alignment positions expressed as serial numbers can be confirmed at the upper part of the alignment results in FIG. 2.

The alignment positions in the present invention comprise conservative and non-conservative positions of nucleotides of the plurality of target nucleic acid sequences that are aligned.

The term used herein, “conservativity” means that, at an alignment position of a plurality of target nucleic acid sequences, the ratio of the number of a same certain type of bases to the total number of bases or the number of a same certain type of bases is a predetermined value or more, the ratio or number of a certain different type of bases to the total number of bases or the number of a particular same type of bases is a predetermined value or less, or a combination of the above. Specifically, the conservativity means that, at the alignment position, the ratio of the number of a same certain type of bases to the total number of bases is 95% or more, 96% or more, 97% or more, 98% or more, or 99% or more, or the number of a same certain type of bases is 60 or less, 50 or less, 40 or less, 30 or less, or 20 or less, or a combination of the above. In addition, the term “conservative position” refers to an alignment position at which nucleotides of a plurality of aligned target nucleic acid sequences exhibit conservativity, and the term “conservative base” indicates one type of bases (i.e., one base) exhibiting conservativity. In the present invention, when the ratio of the number of nucleotides expressed as R, Y, or N due to non-sequencing to the total number of bases or the number of such nucleotides is a predetermined value (specifically, 5%, 4%, 3%, 2%, or 1%, or 60, 50, 40, 30, or 20) or less, such nucleotides are not considered as a different type of bases in determining conservativity.

As used herein, the term “non-conservativity” means that conservativity not exhibited at an alignment position of a plurality of target nucleic acid sequences, and the term means that, at an alignment position of a plurality of target nucleic acid sequences, the ratio of the number of a same certain type of bases to the total number of bases or the number of a same certain type of bases is less than a predetermined vale, the ratio of a certain different type of bases to the total number of bases or the number of a certain different type of bases is more than a predetermined value, or a combination of the above. Specifically, the non-conservativity means that, at the alignment position, the ratio of the number of a same certain type of bases to the total number of bases is less than 99%, less than 98%, less than 97%, less than 96%, or less than 95% or the number of a certain different type of bases is more than 20, more than 30, more than 40, more than 50, or more than 60, or a combination of the above. In addition, the term “non-conservative position” refers to an alignment position at which nucleotides of a plurality of aligned target nucleic acid sequences exhibit non-conservativity, and the term “non-conservative base” indicates two or more types of bases (i.e., two or more bases) exhibiting non-conservativity. In the present invention, when the ratio of the number of nucleotides expressed as R, Y, or N due to non-sequencing to the total number of bases or the number of such a type of nucleotides is more than a predetermined value (specifically, 1%, 2%, 3%, 4%, or 5%, or 20, 30, 40, 50, or 60), such a type of nucleotides are considered as a different type of bases in determining non-conservativity.

In FIG. 2, non-conservative positions are the positions of alignment nos. 12, 22, and 25, and the positions excluding the non-conservative positions and a gap-containing position (the position of alignment no. 33) are conservative positions.

The term used herein, “start position” refers to any one of alignment positions of a plurality of target nucleic acid sequences, which becomes a start point of a region constituting an oligonucleotide stick generated in the present invention, and the start position is specifically a conservative position or a non-conservative position in the alignment positions, more specifically a conservative position, and most specifically the first conservative position of the alignment positions. For example, the position of alignment no. 1 is a start position in FIG. 2.

Step (b): Selecting as an End Position a Position Comprising a Non-Conservative Position within a Predetermined Allowable Number (120)

Then, a position comprising a non-conservative position within a predetermined allowable number from the start position is selected as an end position.

An end position selected in the present invention is any one of alignment positions of a plurality of target nucleic acid sequences which becomes an end point of a region constituting an oligonucleotide stick created in the present invention and a position comprising a non-conservative position within a predetermined allowable number from the start position is selected as an end position. The end position is specifically a conservative position or a non-conservative position of the alignment positions, and more specifically, a conservative position.

The expression used herein “a position comprising a non-conservative position within a predetermined allowable number from the start position is selected as an end position” may mean “a position after a predetermined allowable number or less of non-conservative positions existing from the start position is selected as an end position”.

The predetermined allowable number of non-conservative positions included between the start position and the end position is specifically 1, 2, 3, 4, 5, 6, 7, 8, 9, or 10, but is not limited thereto. More specifically, the predetermined allowable number is 1, 2, 3, 4, or 5.

The term “within a predetermined allowable number” used herein with referring to a non-conservative position means each of a predetermined allowable number or less, and zero is also included therein. For example, when the predetermined allowable number is 3, three or less non-conservative positions means including each of zero, one, two, and three non-conservative positions. In addition, the meaning of including zero non-conservative positions is that only conservative positions are included.

Herein, the number of positions may be exchangeably used with the number of bases. For example, the meaning of including non-conservative positions within a predetermined allowable number is that non-conservative bases within a predetermined allowable number are included.

The term “nucleotide” used herein with referring to the number of positions, the number of bases, and length, may be exchangeably used with “base” or “mer”.

According to an embodiment of the present invention, the end position in step (b) is present in two or more.

For example, FIG. 2 shows a case where the number of non-conservative positions included in the position selected as the end position is within two. In other words, FIG. 2 shows a case where the number of non-conservative positions before positions selected as end positions is two or less. In such a case, the position comprising zero, one, and two non-conservative positions from the position of alignment no. 1 as a start position may be selected as an end position. As for an oligonucleotide stick containing one of the non-conservative positions, each of the positions of alignment nos. 13 to 21 may be selected as an end position.

According to an embodiment of the present invention, the end position in step (b) is a position before the non-conservative position right after the final non-conservative position among non-conservative positions within the predetermined allowable number. The position before the non-conservative position is specifically a position immediately before the non-conservative position, and more specifically, a conservative position right before the non-conservative position. For example, the predetermined allowable number is 2 in the first round of stick generation in FIG. 2, and thus non-conservative positions within two are the position of alignment no. 12 (in cases of one non-conservative position) and the position of alignment no. 22 (in cases of two non-conservative positions), respectively. The non-conservative positions immediately after the non-conservative positions are the position of alignment no. 22 (in cases of one non-conservative position) and the position of alignment no. 25 (in cases of two non-conservative positions), respectively; and the conservative positions immediately before the non-conservative positions are the position of alignment no. 21 (in cases of one non-conservative position) and the position of alignment no. 24 (in cases of two non-conservative positions), and such conservative positions are end positions, respectively. In addition, since non-conservative positions within two include zero non-conservative position, the non-conservative position right after zero non-conservative position is the position of alignment no. 12, which is the first non-conservative position from the start position, and the conservative position right before the non-conservative position is the position of alignment no. 11, so the position of alignment no. 11 is an end position.

### Step (c): Generating Oligonucleotide Stick (130)

Then, an oligonucleotide stick composed of a region from the start position to the end position is generated. The oligonucleotide stick comprises sequence information determined by a plurality of target nucleic acid sequences that are aligned in the region.

The term “generation or creation” used herein with referring to the oligonucleotide stick, does not mean the generation of a material oligonucleotide stick but the generation of sequence information of an oligonucleotide stick.

According to an embodiment of the present invention, the sequence information contains information about conservative and non-conservative positions and the types of conservative and non-conservative bases.

According to an embodiment, the oligonucleotide stick in step (c) is a plurality of oligonucleotide sticks that have the same start position and the same number of non-conservative positions but different end positions.

As described above, when the number of non-conservative positions is within two, positions comprising zero, one, and two non-conservative positions from the position of alignment no. 1 as a start position may be selected as end positions. In oligonucleotide sticks containing one non-conservative position of the created oligonucleotide sticks, all the positions of alignment nos. 13 to 21 may be selected as end positions, and thus, as for oligonucleotide sticks containing one non-conservative position, a plurality of oligonucleotide sticks having the same start position and the same number of non-conservative positions but two or more different end positions may be created.

According to an embodiment of the present invention, the oligonucleotide stick in step (c) is the longest oligonucleotide stick of oligonucleotide sticks comprising only conservative positions or the longest oligonucleotide stick of oligonucleotide sticks having the same number of non-conservative positions.

FIG. 2 shows that the longest oligonucleotide sticks of oligonucleotide sticks having the same start position and the same number of non-conservative positions are created. For example, in the first round of stick generation in FIG. 2, the longest oligonucleotide stick containing zero non-conservative position, that is, only conservative positions, is created by selecting, as an end position, the position of alignment no. 11, which is the conservative position right before the position of alignment no. 12, which is a non-conservative position; the longest oligonucleotide stick containing one non-conservative position is created by selecting, as an end position, the position of alignment no. 21, which is the conservative position right before the position of alignment no. 22, which is a non-conservative position; and the longest oligonucleotide stick containing two non-conservative positions is created by selecting, as an end position, the position of alignment no. 24, which is the conservative position right before the position of alignment no. 25, which is a non-conservative position, so the longest oligonucleotide sticks are created, respectively.

### Step (d): Repeating Generation of Oligonucleotide Stick (140)

Then, the generation of an oligonucleotide stick is repeated by selecting at least one start position different from the start position in step (a).

According to the present invention, a designable region of oligonucleotides for covering a plurality of target nucleic acid sequences is determined in the plurality of target nucleic acid sequences, and thus it is necessary to create a plurality of oligonucleotide sticks containing information about non-conservative positions within a predetermined allowable number having different start positions. Therefore, a procedure of selecting at least one start position different from the start position in step (a), selecting, as an end position, a position comprising non-conservative positions within a predetermined allowable number from the at least one start position, and then creating an oligonucleotide stick composed of a region from the at least one start position to the end position is repeated.

According to an embodiment of the present invention, in step (d), the at least one start position different from the start position in step (a) is selected from positions after non-conservative positions existing after the start position in step (a), specifically, selected from positions right after non-conservative positions existing after the start position in step (a), and more specifically, selected from conservative positions right after non-conservative positions existing after the start position in step (a).

According to an embodiment, in step (d), the at least one start position different from the start position in step (a) is sequentially selected from positions after non-conservative positions existing after the start position in step (a), specifically, sequentially selected from positions right after non-conservative positions exiting after the start position in step (a), and more specifically, sequentially selected from conservative positions right after non-conservative positions existing after the start position in step (a).

For example, in the first round of stick generation of FIG. 2, the start position of the oligonucleotide sticks is the position of alignment no. 1, and start positions of the oligonucleotide sticks created in the next second round of stick generation may be selected from conservative positions (i.e., the positions of alignment nos. 13, 23, and 26) immediately after the non-conservative positions (i.e., the positions of alignment nos. 12, 22, and 25) existing after the start position of the first round of stick generation, and alternatively, may be sequentially selected from the positions of alignment nos. 13, 23, and 26.

In an embodiment, suppose that the start positions are sequentially selected from the positions of alignment nos. 13, 23, and 26. Then, in the second round of stick generation in FIG. 2, the position of alignment no. 13, which is the conservative position right after the position of alignment no. 12, which is a non-conservative position existing after the position of alignment no. 1, which is the start position in the first round of stick generation, is used as a start position, and positions comprising non-conservative positions within two from the position of alignment no. 13 are selected as end positions (specifically, the position of alignment no. 21 in a case of zero non-conservative positions, the position of alignment no. 24 in a case of one non-conservative position, and the position of alignment no. 32 in a case of two non-conservative positions), so secondary oligonucleotide sticks composed of regions from the start position to the end positions are created. In such a manner, tertiary and quaternary oligonucleotide sticks are sequentially created by using the positions of alignment nos. 23 and 26 as start positions for the third and fourth rounds of stick generation.

According to an embodiment of the present invention, the number of predetermined non-conservative positions may be equal or different for repeated rounds, and may be changed by predetermined rules or may be randomly selected within a predetermined range.

According to an embodiment, the oligonucleotide sticks are generated or selected to satisfy at least one (specifically at least two, more specifically at least three, still more specifically at least four, and most specifically at least five) of the following criteria:

(i) a predetermined minimum length of an oligonucleotide stick,

(ii) a gap ratio; wherein when the alignment positions of the plurality of target nucleic acid sequences comprise a gap-containing position, the oligonucleotide sticks are generated by selecting as an end position a position before a gap-containing position having a gap ratio exceeding a predetermined gap ratio, and wherein the gap ratio represents a ratio between the number of gaps and the total number of bases at the gap-containing position and the total number of bases represents the sum of the numbers of existing bases and gaps,

(iii) a base exist ratio (BER) at each position of an oligonucleotide stick; wherein the BER represents a ratio between the sum of the numbers of existing bases and gaps at an alignment position corresponding to each position of an oligonucleotide stick and the total number of sequences that are aligned, and wherein the oligonucleotide stick is selected according to the number of positions each having a BER of less than a predetermined value,

(iv) a GC content; wherein a portion satisfying a predetermined GC content in an oligonucleotide stick is selected, and

(v) amplicon region formation; wherein an amplicon region corresponding to a predetermined length in the 3′ direction from the 5′-end or in the 5′-direction from the 3′-end of an oligonucleotide stick is set, and oligonucleotide sticks included in the amplicon region are selected considering criteria regarding a stick base sum (SBS) and/or respective lengths of the oligonucleotide sticks included in the amplicon region.

According to the present invention, the oligonucleotide sticks may be generated or selected on the basis of criteria (i) to (v) above as generation or selection criteria, in addition to being created on the basis of the number of non-conservative positions. Therefore, criteria (i) to (v) above are both generation criteria and selection criteria. For example, when the criteria (i) to (v) are creation criteria, the oligonucleotide sticks according to the present invention may be created to satisfy at least one of the criteria (i) to (v), in addition to the criterion regarding the number of non-conservative positions, and when the criteria (i) to (v) are selection criteria, the oligonucleotide sticks according to the present invention may be created to satisfy the criterion regarding the number of non-conservative positions and then selected to satisfy at least one of the criteria (i) to (v). Alternatively, at least one of the criteria (i) to (v) may be a creation criterion, and the other criteria may be selection criteria. Specifically, criteria (i) and (ii) of the criteria (i) to (v) may be creation criteria, and the criteria (iii) to (v) may be selection criteria.

According to an embodiment of the present invention, the oligonucleotide sticks may be created to satisfy at least one of the criteria (i) and (ii). Specifically, the oligonucleotide sticks may be created to satisfy criterion (i).

According to an embodiment, the oligonucleotide sticks may be selected to satisfy at least one of the criteria (iii) and (iv).

According to an embodiment of the present invention, the oligonucleotide sticks may be selected to satisfy criterion (v).

According to an embodiment, the oligonucleotide sticks may be created to satisfy at least one of the criteria (i) and (ii); selected to satisfy at least one of the criteria (iii) and (iv), and selected to satisfy the criterion (v).

As for criterion (i) regarding a predetermined minimum length of an oligonucleotide stick of the creation or selection criteria, a predetermined minimum length of an oligonucleotide stick may be selected considering the length of an oligonucleotide to be designed from a designable region determined on the basis of the oligonucleotide sticks. The predetermined minimum length of an oligonucleotide stick may be specifically 5, 10, 15, 20, 25, 30, or 35 nucleotides, but is not limited thereto. For example, the predetermined minimum length of an oligonucleotide stick may be one selected from 5 nucleotides to 100 nucleotides.

The criterion regarding a predetermined minimum length of an oligonucleotide stick is not particularly required. Since a designable region of oligonucleotides is determined from alignment positions of a plurality of target nucleic acid sequences on the basis of oligonucleotide sticks, a longer oligonucleotide stick is preferable as long as the oligonucleotide stick satisfies a creation criterion regarding the number of non-conservative positions.

In FIG. 2, a predetermined minimum length of an oligonucleotide stick as a creation or selection criterion is 20 nucleotides. In FIG. 2, the oligonucleotide sticks containing zero non-conservative positions in the first round of stick generation, and the oligonucleotide sticks containing zero and one non-conservative position in the second round of stick generation are treated as “dropouts” since such oligonucleotide sticks do not satisfy the predetermined minimum length.

The oligonucleotide sticks of the present invention may be created or selected to satisfy criterion (ii) regarding a gap ratio.

According to an embodiment of the present invention, the gap represents a non-homologous position existing in a sequence of target nucleic acid sequences aligned according to homology of the plurality of target nucleic acid sequences.

According to an embodiment, the gap represents a portion where a base is absent in a sequence of the aligned target nucleic acid sequences.

Herein, a gap is distinguished from a partial sequence having a portion where a base is absent in one end of a sequence (a miss portion). For example, in FIG. 2, the sequence 2 has a portion where a base is absent, that is, a gap, at the position of alignment no. 33, according to the homology in the aligning procedure of the plurality of sequences. The sequence 5 is a partial sequence having portions where the positions of alignment nos. 27 to 35, corresponding to the 3′-end of the sequence, are vacant (miss portions). As for a partial sequence, the conservativity at an alignment position is determined as follows: Specifically, in FIG. 2, as for the miss portion of the sequence 5 at the position of alignment no. 27, the miss portion of the sequence 5 is not considered when the conservativity of the position of alignment no. 27 is determined. That is, the C base accounts for 100% at the position of alignment no. 27, and thus the position of alignment no. 27 exhibits conservativity.

When a gap exists in the sequence of any target nucleic acid sequence of a plurality of aligned target nucleic acid sequences, an alignment position at which the gap exists is referred to as a gap-containing position, and the alignment positions of the plurality of target nucleic acid sequences may include a gap-containing position, in addition to conservative and non-conservative positions. Specifically, referring to FIG. 2, a gap exists at the position of alignment no. 33 in the sequence of the sequence 2, and thus the position of alignment no. 33 is a gap-containing position.

The gap ratio represents a ratio of the number of gaps to the total number of bases at a gap-containing position, and the total number of bases represents the sum of the number of existing bases and the number of gaps. For example, the gap ratio at the position of alignment no. 33 is 25%, which is a ratio of the number of the gap of Sequence 2 to the sum of the number of A bases of the sequences 1, 3, and 4 and the number of gaps of the sequence 2. In other words, when the gap ratio at the position of alignment no. 33 is calculated, the miss portion of the sequence 5 is not considered.

The oligonucleotide sticks may be created by using, as an end position, a position before a gap-containing position having a gap ratio exceeding a predetermined gap ratio. The predetermined gap ratio is specifically, 0.5%, 1%, 5%, 10%, 15%, 25%, 50%, 60%, or 75%, but is not limited thereto. For example, the predetermined gap ratio may be selected from 0.5-90%.

According to an embodiment of the present invention, a position before the gap-containing position is the position immediately before the gap-containing position. Specifically, the position immediately before the gap-containing position is a conservative position.

Suppose that the gap ratio is a generation criterion and the predetermined gap ratio is 1%. Then, as for an oligonucleotide stick containing two non-conservative positions in the second round of stick generation in FIG. 2, the gap ratio at the position of alignment no. 33, which is a gap-containing position, is 25%, which exceeds 1%, the predetermined gap ratio. Therefore, the oligonucleotide stick is created by using, as an end position, the position of alignment no. 32 right before the gap-containing position.

Alternatively, when the gap ratio is a selection criterion and the predetermined gap ratio is 1%, an oligonucleotide stick containing two non-conservative positions in the second round of stick generation in FIG. 2 may be created by using the position of alignment no. 35 as an end position, but may be selected by using, as an end position, the position of alignment no. 32, which is the position right before the gap-containing position having a gap ratio exceeding the predetermined gap ratio.

A position before a gap-containing position may be selected as an end position and a position after a gap-containing position may be selected as a start position, specifically, the start position is the position immediately after the gap-containing position, and more specifically, the start position is the conservative position immediately after the gap-containing position. For example, in FIG. 2, the position of alignment no. 34, which is the conservative position right after the position of alignment no. 33, which is a gap-containing position, may also be selected as a start position.

In short, in the present embodiment, a gap-containing position having a predetermined gap ratio is not contained in the oligonucleotide stick.

According to an embodiment, when a position at which a gap exists has a predetermined gap ratio even though the other bases excluding the gap have conservativity or non-conservativity, the position at which the gap exists is a gap-containing position but not a conservative position or non-conservative position. That is, a gap has priority over conservativity or non-conservativity.

The oligonucleotide stick of the present invention may be created or selected to satisfy criterion (iii) regarding a base exist ratio (BER) at each position of the oligonucleotide stick.

The BER represents a ratio between the sum of the numbers of existing bases and gaps at an alignment position corresponding to each position of the oligonucleotide stick and the total number of aligned sequences. Specifically, the BER represents a ratio between the sum of the numbers of existing bases and gaps at an alignment position corresponding to each position of the oligonucleotide stick to the total number of aligned sequences, or a ratio of the total number of aligned sequences to the sum of the numbers of existing bases and gaps at an alignment position corresponding to each position of the oligonucleotide stick. More specifically, the BER represents a ratio of the sum of the numbers of existing bases and gaps at an alignment position corresponding to each position of the oligonucleotide stick to the total number of aligned sequences. For example, in the second round of stick generation in FIG. 2, the BERs at the positions of alignment nos. 26 and 27 corresponding to the oligonucleotide stick containing two non-conservative positions are 100% and 80%, respectively. For example, suppose that the number of aligned sequences is 200 and there is one gap, 190 A bases, and 9 miss portions at the position of alignment no. 33. Then, the gap ratio at the position of alignment no. 33 is a ratio of 1 to 191, that is, 0.5%, and the BER at the same position is a ratio of 191 to 200, that is, 96%.

According to an embodiment of the present invention, the number of gaps may be excluded in the calculation of BER.

The reason why the BER at each position of the oligonucleotide stick is selected as a creation criterion or a selection criterion in the present invention is to create or select an oligonucleotide stick from a portion where as many sequences as possible are aligned.

The oligonucleotide stick is created or selected according to the number of positions each having a BER of less than a predetermined value. The predetermined value of BER is 50%, 40%, 30%, 20%, or 10%, and the number of positions is 20 mers or less, 15 mers or less, 10 mers or less, or 5 mers or less, but are not limited thereto. For example, the predetermined value of BER may be selected from 5% to 70%, and the number of positions as a standard for “or less” may be selected from 5 mers to 20 mers.

Suppose that the number of positions each having a BER of less than 30% is 10 mers or less as a creation or selection criterion. Then, as for three types of oligonucleotide sticks in the first round of stick generation in FIG. 2, all the three types of oligonucleotide sticks are neither created nor selected if the BERs at the positions of alignment nos. 1 to 11 are 20% and the BERs at the positions of alignment nos. 12 to 24 are 100%, but all the three types of oligonucleotide sticks can be created or selected if the BERs at the positions of alignment nos. 1 to 10 are 20% and the BERs at the positions of alignment nos. 11 to 24 are 100% (However, the oligonucleotide sticks containing zero non-conservative positions in the first round of stick generation are dropped out according to the criterion regarding a predetermined minimum length of an oligonucleotide stick.

The oligonucleotide sticks of the present invention may be created or selected to satisfy criterion (iv) regarding a GC content. In addition, criterion (iv) regarding a GC content may be considered in the creation step or selection step. Specifically, a portion satisfying a predetermined GC content in the oligonucleotide stick is selected. A portion having a GC content of more than 5%, more than 10%, more than 15%, more than 20%, more than 25%, or more than 30% in the unit of the minimum length of criterion (i) in an oligonucleotide stick is created or selected. The standard for “more than” with respect to a GC content may be selected from 3% to 50%.

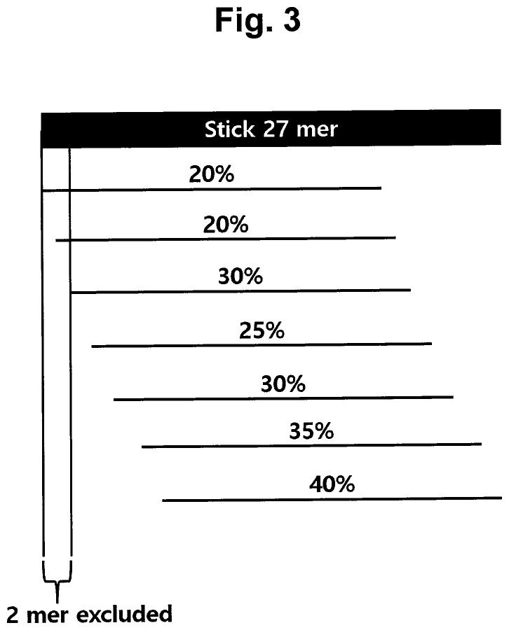

FIG. 3 shows a procedure of selecting a portion satisfying a predetermined GC content in a oligonucleotide stick generated according to an embodiment of the present invention.

Suppose that the created oligonucleotide stick is 27 mers in length and a predetermined GC content of more than 20% and a minimum stick length of 20 mers are needed. Then, when it is investigated whether the criterion regarding a predetermined GC content is satisfied in the 20-mer unit of the stick in FIG. 3, the predetermined GC content is satisfied from the third position of the 27-mer stick, and therefore, the other portions excluding 2 mers corresponding to the first and second positions are selected.

In addition, when an oligonucleotide stick created or selected according to criterion (iv) regarding a GC content does not satisfy criterion (i) regarding a minimum length, such an oligonucleotide stick is dropped out.

The oligonucleotide sticks of the present invention may be created or selected to satisfy criterion (v) regarding amplicon region formation.

The reason why the criterion regarding amplicon region formation is set as a creation or selection criterion in the present invention is to check whether primers can be combined since amplification should be made by a primer pair designed from a designable region determined on the basis of oligonucleotide sticks.

According to the present invention, an amplicon region corresponding to a predetermined length in the 3′ direction from the 5′-end or in the 5′-direction from the 3′-end of an oligonucleotide stick is set, and oligonucleotide sticks included in the amplicon region are selected considering the criterion regarding a stick base sum (SBS) of oligonucleotide sticks included in the amplicon region and/or a length of each of the oligonucleotide sticks.

The predetermined length of the amplicon region may be selected from 150-450 bases, 200-400 bases, 250-400 bases, or 300-400 bases, but is not limited thereto.

It is considered whether the stick base sum (SBS) of the oligonucleotide sticks included in the amplicon region satisfies more than 50 bases, more than 70 bases, more than 80 bases, more than 100 bases, more than 120 bases, more than 150 bases, more than 170 bases, or more than 200 bases. According to an embodiment, in the calculation of the stick base sum of the oligonucleotide sticks, an overlapping base is considered only once when two or more oligonucleotide sticks have the overlapping base. For example, suppose that the oligonucleotide sticks 1 and 2 included in the amplicon region 1 have 100 bases and 50 bases, respectively, and have 10 overlapping bases. Then, the SBS of the oligonucleotide sticks 1 and 2 is 140 bases.

As for respective lengths of oligonucleotide sticks included in the amplicon region, it is considered whether the length of at least one of the oligonucleotide sticks satisfies a predetermined length (specifically, 70 bases, 90 bases, 100 bases, 120 bases, or 140 bases) or more or at least two thereof satisfy a predetermined length (specifically, 20 bases, 30 bases, 40 bases, 50 bases, or 60 bases) or more.

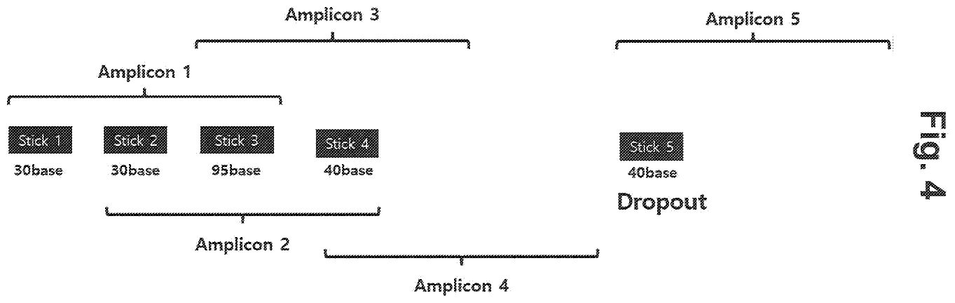

FIG. 4 shows a procedure of selecting oligonucleotide sticks, passing through criteria (i) to (iv), using an amplicon filter (amplicon region forming ability) according to an embodiment of the present invention.

In FIG. 4, the amplicon regions 1 to 5 are shown by setting an amplicon region corresponding to a predetermined length in the 3′-direction from the 5′-end of each of the oligonucleotide sticks 1 to 5. First, the oligonucleotide stick 5 included in the amplicon region 5 is dropped out since the oligonucleotide stick fails to combine with another oligonucleotide. For example, it is checked whether the oligonucleotide sticks included in the amplicon regions 1 and 2 of the amplicon regions 1 to 4 in FIG. 4 are selected according to the criterion regarding amplicon region formation. Here, it is considered whether the SBS is more than 150 bases and the length of at least one of the oligonucleotide sticks is 100 bases or more or the lengths of at least two of the oligonucleotide sticks are 40 bases or more. In FIG. 4, the amplicon region 1 includes the oligonucleotide stick 1 with 30 bases, the oligonucleotide stick 2 with 30 bases, and the oligonucleotide stick 3 with 95 bases, and thus the SBS is 155 bases, which satisfies the criterion regarding SBS, but the respective lengths of the oligonucleotide sticks fail to satisfy that the length of at least one oligonucleotide stick is 100 bases or more or the lengths of at least two oligonucleotide sticks are no 40 bases or more. Therefore, the oligonucleotide sticks 1 to 3 included in the amplicon region 1 fail to satisfy the criterion regarding amplicon region formation, and thus are not selected. In FIG. 4, the amplicon region 2 includes the oligonucleotide stick 2 with 30 bases, the oligonucleotide stick 3 with 95 bases, and the oligonucleotide stick 4 with 40 bases, and thus the SBS is 165 bases, which satisfies the criterion regarding SBS, and the respective lengths of the oligonucleotide sticks fail to satisfy that the length of at least one oligonucleotide stick is 100 bases or more, but satisfy that the lengths of at least two oligonucleotide sticks are 40 bases or more. Therefore, the oligonucleotide sticks 2 to 4 included in the amplicon region 2 satisfy the criterion regarding amplicon region formation, and thus are selected. Meanwhile, all of the oligonucleotide sticks 1 to 3 included in the amplicon region 1 are not dropped out, and only the oligonucleotide stick 1 not included in the amplicon region 2 is not selected.

According to an embodiment of the present invention, the creation or selection criteria may further include (vi) a match ratio of a predetermined value between an oligonucleotide stick and a nucleic acid sequence of a non-target nucleic acid molecule.

As used herein, the term “non-target nucleic acid molecule” has a contrary concept to the above-described target nucleic acid molecule, and refers to a nucleic acid molecule that should not be detected in the detection procedure of a target nucleic acid molecule regardless of the homology with a sequence of the target nucleic acid molecule. The non-target nucleic acid molecule may be used exchangeably with an exclusive nucleic acid sequence.

According to an embodiment, the non-target nucleic acid molecule may be a molecule other than a target nucleic acid molecule. Alternatively, the non-target nucleic acid molecule may be selected. According to an embodiment, the non-target nucleic acid sequence may be a nucleic acid sequence other than target nucleic acid sequences. Alternatively, the non-target nucleic acid sequence may be selected.

The term “match” used herein means that when two sequences to be compared have identical orientation, two bases corresponding to the same position of the two sequences are identical, and that when two sequences have different orientations, two bases corresponding to the two sequences are complementary.

The oligonucleotide sticks of the present invention contain sequence information determined by a plurality of target nucleic acid sequences that are aligned, that is, information about conservative and non-conservative positions and the types of conservative and non-conservative bases. Therefore, a comparison is made of whether an oligonucleotide stick having such sequence information is matched to a nucleic acid sequence of a non-target nucleic acid molecule.

The predetermined value of the match ratio may be selected from 50% to 100%. For example, when the predetermined value of the match ratio is 100%, that is, when an oligonucleotide stick having sequence information and a nucleic acid sequence of a non-target nucleic acid molecule are analyzed to be 100% matched to each other, such an oligonucleotide stick is neither created nor selected, and other oligonucleotide sticks showing a match ratio of less than 100% are created or selected.

According to an embodiment, the creation or selection of an oligonucleotide stick may be determined considering amplicon region forming ability of the oligonucleotide stick as well as the match ratio between the oligonucleotide stick and a non-target nucleic acid sequence. For example, when the match ratios between oligonucleotide sticks having sequence information included in amplicon regions and a nucleic acid sequence of a non-target nucleic acid molecule are analyzed, all of oligonucleotide sticks included in an amplicon region including at least one oligonucleotide stick having a match ratio of less than 100% are created or selected, and oligonucleotide sticks having a match ratio of 100% included in an amplicon region not including at least one oligonucleotide stick having a match ratio of less than 100% are not created or selected.

According to an embodiment of the present invention, the oligonucleotide sticks are ranked according to at least one (specifically, at least two, and most specifically three) of the following priority items:

(i) a ratio of the number of bases of an oligonucleotide stick to the number of non-conservative bases of the oligonucleotide stick; the larger the ratio, the higher the priority;

(ii) an average base exist ratio (BER) of an oligonucleotide stick; the larger the average BER, the higher the priority, and

(iii) the number of amplicon regions in which one oligonucleotide stick is included; the larger the number, the higher the priority.

The oligonucleotide sticks generated (or selected) in the present invention may be ranked according to the priority items.

The present embodiment may be implemented considering the degree of creation (or selection) of oligonucleotide sticks or may be implemented independently without considering the degree.

In the implementation of the present embodiment considering the degree of creation (or selection) of oligonucleotide sticks, the present embodiment is carried out when all of the following standards are satisfied: (i) the stick base sum (SBS) of oligonucleotide sticks is a predetermined value or more (absolute standard); and (ii) the ratio of SBS to the number of alignment positions having a BER of a predetermined value or more among alignment positions of a plurality of target nucleic acid sequences is a predetermined value or more (relative standard). The predetermined value of SBS in standard (i) is specifically 300, 400, 500, 600, 700, 800, or 900 bases; the predetermined value of BER in standard (ii) is 10, 20, 30, 40, or 50% and the predetermined value of the ratio of SBS in standard (ii) is 30, 40, 50, 60, 70, or 80%, but are not limited thereto.

For example, the predetermined value of SBS in standard (i) may be selected from 300 to 900 bases; the predetermined value of BER in standard (ii) may be selected from 10 to 50% and the predetermined value of the ratio of SBS in standard (ii) may be selected from 30 to 80%.

The term used herein, “average base exist ratio (BER)” refers to an average value of base exist ratios (BER) at respective positions of an oligonucleotide stick.

In an embodiment, the oligonucleotide sticks may be given scores and ranked according to at least one (specifically, at least two, and most specifically three) of the priority items. For example, when given scores and ranked on the basis of priority item (i), an oligonucleotide stick is ranked so that the score and priority is high as the ratio of the number of bases of the oligonucleotide stick to the number of non-conservative bases of the oligonucleotide stick is large.

In an embodiment, when given scores and ranked according to at least two of the priority items, the sum of the scores of respective items is found, and the larger the sum, the higher the rank of the oligonucleotide stick.

In an embodiment, the scores may be given according to the priority items by using different weights of the scores for the priority items. For example, the scores may be given by increasing the weight in order of priority items (i), (ii), and (iii).

According to an embodiment of the present invention, the method between steps (d) and (e), further comprises arranging amplicon regions according to the sum of the numbers of bases of oligonucleotide sticks ranked in a predetermined ranking or more among the oligonucleotide sticks included in the amplicon regions; selecting amplicon regions ranked in a predetermined ranking or more among the arranged amplicon regions; and selecting oligonucleotide sticks included in the selected amplicon regions.

When the oligonucleotide sticks created (or selected) in the present invention are ranked according to priority items, amplicon regions are arranged according to the sum of the numbers of bases of oligonucleotide sticks ranked in a predetermined ranking or more, amplicon regions ranked in a predetermined ranking or more are selected among these, and oligonucleotide sticks included in the selected amplicon regions are selected.

In the step of arranging amplicon regions, the predetermined rankings of the oligonucleotide sticks considering the sum of the numbers of bases are the top 50%, the top 40%, the top 30%, the top 20%, the top 10%, or the top 5%, but the predetermined rankings may be selected considering the number of created or selected oligonucleotide sticks. As for the sum of the numbers of bases of the oligonucleotide sticks considered in the step of arranging amplicon regions, the larger the sum, the higher the priority of the amplicon region. For example, suppose that, in consideration of the sum of the numbers of bases, the predetermined rankings of oligonucleotide sticks are the top 30% or more, the amplicon region 1 includes the oligonucleotide stick 2 (50 bases) ranked on the top 5% and the oligonucleotide stick 3 (60 bases) ranked on the top 10%, and the amplicon region 3 includes the oligonucleotide stick 5 (80 bases) ranked on the top 25% and the oligonucleotide stick 7 (70 bases) ranked on the top 30%. Then, the rankings of the oligonucleotide sticks included in the amplicon region 3 are low, but the sum of the numbers of bases of the oligonucleotide sticks included in the amplicon region 3 is more than that of the oligonucleotide sticks included in the amplicon region 1, and therefore, the amplicon region 3 has a higher ranking than the amplicon region 1 in the arrangement of amplicon regions.

In the step of selecting amplicon regions, the predetermined rakings are the top 70%, the top 60%, the top 50%, the top 40%, or the top 30%, but the predetermined rankings may be selected considering the number of created or selected oligonucleotide sticks.

The oligonucleotide sticks included in the amplicon regions selected in such a manner are selected, and may be used to determine a designable region of oligonucleotides.

### Step (e): Determining Designable Region of Oligonucleotide (150)

Last, regions in an alignment of the plurality of target nucleic acid sequences, which correspond to the regions of the oligonucleotide sticks, are determined as a designable region of oligonucleotides.

When the oligonucleotide sticks have no overlapping areas, respective areas of the oligonucleotide sticks correspond to a region of the oligonucleotide sticks, and when the oligonucleotide sticks have overlapping areas, a region linking the overlapping areas corresponds to a region of the oligonucleotide sticks.

The term “oligonucleotide” as used herein refers to a linear oligomer of natural or modified monomers or linkages, including deoxyribonucleotides and ribonucleotides, capable of specifically hybridizing with a target nucleotide sequence, whether occurring naturally or produced synthetically. The oligonucleotide is particularly single stranded for maximum efficiency in hybridization. Particularly, the oligonucleotide is an oligodeoxyribonucleotide. The oligonucleotide of this invention can be comprised of naturally occurring dNMP (i.e., dAMP, dGM, dCMP and dTMP), nucleotide analogs, or nucleotide derivatives. The oligonucleotide can also include ribonucleotides. The oligonucleotide may include nucleotides with backbone modifications such as peptide nucleic acid (PNA) (M. Egholm et al., Nature, 365:566-568 (1993)), locked nucleic acid (LNA) (WO1999/014226), bridged nucleic acid (BNA) (WO2005/021570), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine. Especially, the term “oligonucleotide” used herein is a single strand composed of deoxyribonucleotides. Specifically, the oligonucleotide is a primer and/or a probe.

The term “primer” as used herein refers to an oligonucleotide, which is capable of acting as a point of initiation of synthesis when placed under conditions in which synthesis of primer extension product which is complementary to a target nucleic acid sequence is induced, i.e., in the presence of nucleotides and an agent for polymerization, such as DNA polymerase, and at a suitable temperature and pH. The primer should be long enough to prime the synthesis of the extension product in the presence of an agent for polymerization. The suitable length of the primer depends on a plurality of factors, such as temperature, a field of application, and a primer source.

The primer may be have a length of, for example, 10-100 nucleotides, 10-80 nucleotides, 10-50 nucleotides, 10-40 nucleotides, 10-30 nucleotides, 15-100 nucleotides, 15-80 nucleotides, 15-50 nucleotides, 15-40 nucleotides, 15-30 nucleotides, 20-100 nucleotides, 20-80 nucleotides, 20-50 nucleotides, 20-40 nucleotides, or 20-30 nucleotides. When the primer is the DPO primer developed by the present applicant (see U.S. Pat. No. 8,092,997), the descriptions of the length of DPO primer disclosed in the patent document are incorporated herein by reference.

The term used herein “probe” refers to a single-stranded nucleic acid molecule containing a portion or portions that are complementary to a target nucleic acid sequence. The probe may also contain a label capable of generating a signal for target detection.

The probe may be have a length of, for example, 10-100 nucleotides, 10-80 nucleotides, 10-50 nucleotides, 10-40 nucleotides, 10-30 nucleotides, 15-100 nucleotides, 15-80 nucleotides, 15-50 nucleotides, 15-40 nucleotides, 15-30 nucleotides, 20-100 nucleotides, 20-80 nucleotides, 20-50 nucleotides, 20-40 nucleotides, or 20-30 nucleotides in length. When the probe is a tagging probe, descriptions of the length are applied to a targeting portion of the tagging probe. The tagging portion of the tagging probe may have a length of, for example, may be 7-48 nucleotides, 7-40 nucleotides, 7-30 nucleotides, 7-20 nucleotides, 10-48 nucleotides, 10-40 nucleotides, 10-30 nucleotides, 10-20 nucleotides, 12-48 nucleotides, 12-40 nucleotides, 12-30 nucleotides, or 12-20 nucleotides, but is not limited thereto.

Oligonucleotides that can be designed from the determined designable region may have a conventional primer and probe structure consisting of sequences that are hybridized with a target nucleic acid sequence. Alternatively, the oligonucleotides may have a unique structure through structural modification thereof. For example, the oligonucleotides may have structures of Scorpion primer, Molecular beacon probe, Sunrise primer, HyBeacon probe, tagging probe, DPO primer or probe (WO 2006/095981), and PTO probe (WO 2012/096523).

The oligonucleotide may be a modified oligonucleotide, such as a degenerate base-containing oligonucleotide and/or a universal base-containing oligonucleotide, in which degenerate bases and/or universal bases are introduced into a conventional primer or probe. As used herein, the terms “conventional primer”, “conventional probe”, and “conventional oligonucleotide” refer to a common primer, probe, and oligonucleotide into which a degenerate base or non-natural base is not introduced. According to an embodiment of the present invention, at least 50%, at least 60%, at least 70%, at least 80%, at least 90%, or at least 95% of the degenerate base-containing oligonucleotides or universal base-containing oligonucleotides are non-modified oligonucleotides. According to an embodiment of the present invention, the number of degenerate bases or universal bases introduced into the conventional oligonucleotide is specifically 7 or less, 5 or less, 4 or less, 3 or less, or 2 or less. In addition, the use ratio of degenerate bases and/or universal bases introduced into the conventional oligonucleotide is specifically 25% or less, 20% or less, 18% or less, 16% or less, 14% or less, 12% or less, 10% or less, 8% or less, or 6% or less. The use ratio of degenerate bases or universal bases represents a ratio of degenerate bases or universal bases among all nucleotides of the oligonucleotide into which degenerate bases or universal bases are introduced. The degenerate bases include various degenerate bases known in the art as follows: R: A or G; Y: C or T; S: G or C; W: A or T; K: G or T; M: A or C; B: C or G or T; D: A or G or T; H: A or C or T; V: A or C or G; N: A or C or G or T. The universal bases include various universal bases known in the art as follows: deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitropyrrole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, 2′-O-methoxyethyl 3-nitropyrrole, and combinations thereof. More specifically, the universal bases are deoxyinosine, inosine, or combinations thereof.

According to an embodiment of the present invention, the base introduced for a maximum target coverage is a degenerate base. The degenerate oligonucleotides include a plurality of oligonucleotides represented by degenerate oligonucleotides. Unless especially stated otherwise herein, the degenerate oligonucleotide represents a subgroup comprising a plurality of oligonucleotides represented by degenerate oligonucleotides, but not a single oligonucleotide.

The term used herein, “designable region” refers to a region which is used for being capable of designing an oligonucleotide (primer and/or probe) in a plurality of target nucleic acid sequences. According to an embodiment of the present invention, the designable region is a conservative region containing a sequence that is conservatively maintained across different organisms, that is, a conservative sequence. A conservative region which is a biologically very meaningful portion represents a portion where sequences are similar or identical in different nucleic acid molecules between different organisms from each other. The conservative region is used as a very important indicator for phylogenetic studies and is also used as a probing portion when different organisms are detected in a multiplex manner.

According to an embodiment of the present invention, the designable region is a designable region of oligonucleotides that permits to exhibit a maximum target coverage for the plurality of target nucleic acid sequences with one primer pair and/or one probe.

According to an embodiment, oligonucleotides with the same bases excluding bases introduced for a maximum target coverage (e.g., degenerate bases) may be treated as one oligonucleotide. According to an embodiment of the present invention, primers or probes with the same bases excluding bases (e.g., degenerative bases) introduced for a maximum target coverage may be treated as one primer or one probe.

According to an embodiment of the present invention, one primer pair and/or one probe may be expressed as one oligonucleotide group.

According to the present embodiment, when a designable region is determined on the basis of oligonucleotide sticks containing conservative m positions within a predetermined allowable number, it means that a plurality of target nucleic acid sequences to be amplified or detected by the oligonucleotides have a sequence similarity enough to be covered by one primer pair and/or one probe.

According to an embodiment of the present invention, the method of the present invention is performed by computer-implemented methods. A storage medium, a device, and a computer program for performing the above described method of the present invention on a computer will be described in detail as below.

### Storage Medium, Device, Program

In another aspect of the present invention, there is provided a computer readable storage medium containing instructions to configure a processor to perform a method for determining a designable region of oligonucleotides in a plurality of target nucleic acid sequences having sequence similarity, the method comprising: (a) selecting a start position from alignment positions of a plurality of target nucleic acid sequences; wherein the alignment positions comprise a conservative position and a non-conservative position of nucleotides of the plurality of target nucleic acid sequences that are aligned, the conservative position has one type of bases exhibiting conservativity, and the non-conservative position has two or more types of bases exhibiting non-conservativity; (b) selecting as an end position a position comprising a non-conservative position within a predetermined allowable number from the start position; (c) generating an oligonucleotide stick composed of a region from the start position to the end position; wherein the oligonucleotide stick comprises sequence information determined by a plurality of target nucleic acid sequences that are aligned in the region; (d) repeating the generation of an oligonucleotide stick by selecting at least one start position different from the start position in step (a); and (e) determining as a designable region of oligonucleotides regions in an alignment of the plurality of target nucleic acid sequences, which correspond to the regions of the oligonucleotide sticks.

In still another aspect of the present invention, there is provided a computer program to be stored on a computer readable storage medium, to configure a processor to perform a method for determining a designable region of oligonucleotides in a plurality of target nucleic acid sequences having sequence similarity, the method comprising: (a) selecting a start position from alignment positions of a plurality of target nucleic acid sequences; wherein the alignment positions comprise a conservative position and a non-conservative position of nucleotides of the plurality of target nucleic acid sequences that are aligned, the conservative position has one type of bases exhibiting conservativity, and the non-conservative position has two or more types of bases exhibiting non-conservativity; (b) selecting as an end position a position comprising a non-conservative position within a predetermined allowable number from the start position; (c) generating an oligonucleotide stick composed of a region from the start position to the end position; wherein the oligonucleotide stick comprises sequence information determined by a plurality of target nucleic acid sequences that are aligned in the region; (d) repeating the generation of an oligonucleotide stick by selecting at least one start position different from the start position in step (a); and (e) determining as a designable region of oligonucleotides regions in an alignment of the plurality of target nucleic acid sequences, which correspond to the regions of the oligonucleotide sticks.

In another aspect of the present invention, there is provided a device for determining a designable region of oligonucleotides in a plurality of target nucleic acid sequences having sequence similarity, comprising (a) a computer processor, and (b) a computer readable storage medium of the present method coupled to the computer processor.

Since the storage medium, the device, and the computer program of the present invention are intended to perform the present methods described hereinabove in a computer, the common descriptions between them are omitted in order to avoid undue redundancy leading to the complexity of this specification.

The program instructions are operative, when performed by the processor, to cause the processor to perform the method of the present invention described above. The program instructions for performing a method for determining a designable region of oligonucleotides may comprise the following instructions: (i) an instruction to select a start position from alignment positions of a plurality of target nucleic acid sequences; (ii) an instruction to select as an end position a position comprising a non-conservative position within a predetermined allowable number from the start position; (iii) an instruction to generate an oligonucleotide stick composed of a region from the start position to the end position; (iv) an instruction to repeat the generation of an oligonucleotide stick by selecting at least one start position different from the start position in in instruction (i); and (v) an instruction to determine (e.g., display on an out device) as a designable region of oligonucleotides regions in an alignment of the plurality of target nucleic acid sequences, which correspond to the regions of the oligonucleotide sticks.

The method of the present invention is implemented in a processor, and the processor may be a processor in a stand-alone computer, a network attached computer, or a data acquisition device such as a real-time PCR machine.

The types of the computer readable storage medium include various storage medium known in the art, such as CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory, and web server, but are not limited thereto.

The determined designable region of oligonucleotides may be provided in a variety of ways. For example, the designable region of oligonucleotides may be provided to a separate system, such as a desktop computer system, via a network connection (e.g., LAN, VPN, intranet, and internet) or a direct connection (e.g., USB or other direct wired or wireless connection), or provided on a portable medium, such as CD, DVD, floppy disk, or portable HDD. Similarly, the designable region of oligonucleotides may be provided to a server system via a network connection (e.g., LAN, VPN, internet, intranet, and wireless communication network) to a client, such as a notebook or a desktop computer system.

The instructions to configure the processor to perform the present invention may be included in a logic system. The instructions may be downloaded and stored in a memory module (e.g., hard drive or other memory such as a local or attached RAM or ROM), although the instructions can be provided on any software storage medium, such as portable HDD, USB, floppy disk, CD and DVD. A computer code for implementing the present invention may be implemented in a variety of coding languages, such as C, C++, Java, Visual Basic, VBScript, JavaScript, Perl, and XML. In addition, a variety of languages and protocols may be used in external and internal storage and transmission of data and commands according to the present invention.

The computer processor may be constructed in such a manner that a single processor can make several performances. Alternatively, the processor unit may be constructed in such a manner that several processors make several performances, respectively.

### II. Method for Determining Designable Region of Oligonucleotide (the Second Aspect)

In one aspect of the present invention, there is provided a method for determining a designable region of oligonucleotides in a plurality of target nucleic acid sequences having sequence similarity, comprising:

(a) selecting a start position from alignment positions of a plurality of target nucleic acid sequences;

(b) selecting as an end position a position having the minimum number of sequence patterns within the predetermined allowable number of sequence patterns from the positions located a predetermined length or more apart from the start position; wherein the number of sequence patterns is determined by a plurality of target nucleic acid sequences that are aligned,

(c) generating an oligonucleotide stick composed of a region from the start position to the end position, wherein the oligonucleotide stick comprises the number of sequence patterns and sequence pattern information determined by a plurality of target nucleic acid sequences that are aligned in the region;

(d) repeating the generation of an oligonucleotide stick by selecting at least one start position different from the start position in step (a); and

(e) determining as a designable region of oligonucleotides regions in an alignment of the plurality of target nucleic acid sequences, which correspond to the regions of the oligonucleotide sticks.

A second aspect of the present invention relates to a method in which oligonucleotide sticks having sequence information about the number of sequence patterns are generated from alignment positions of a plurality target nucleic acid sequences and then a designable region of oligonucleotides is determined on the basis of the oligonucleotide sticks. The method according to the second aspect of the present invention is referred to as a pattern stick manner, and as used in the method according to the second aspect of the present invention, the terms “oligonucleotide stick” and “pattern stick” may be exchangeably used with each other.

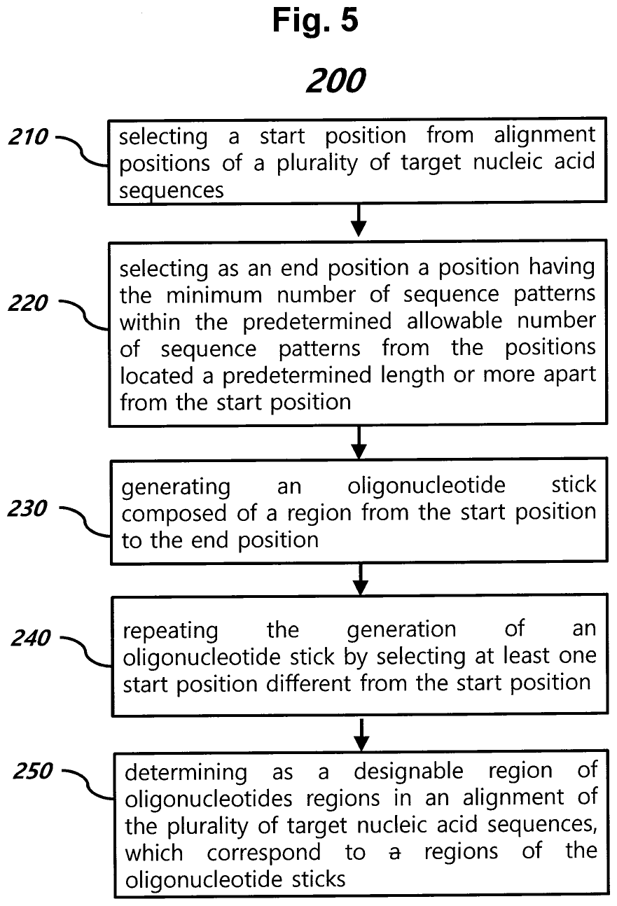

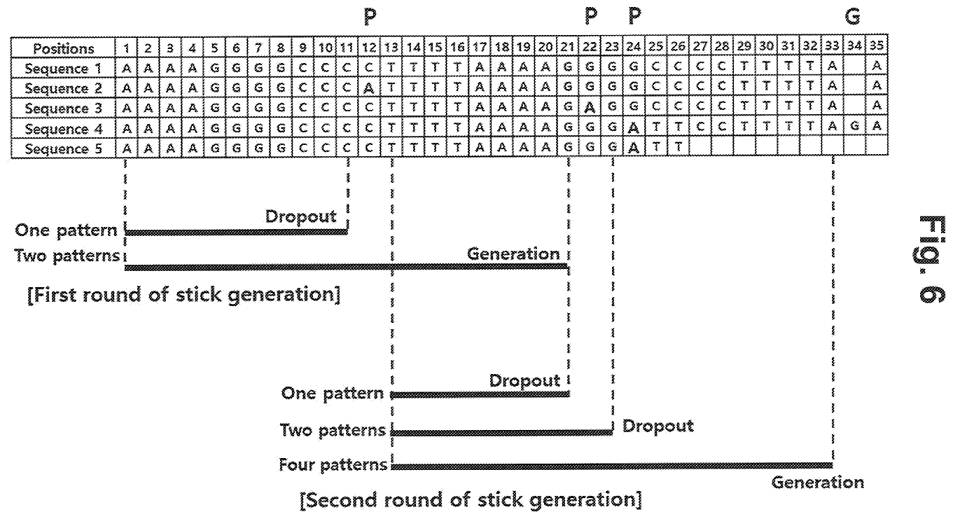

FIG. 5 is a flow diagram of steps for implementing a second aspect of the present invention according to an embodiment of the present invention, and FIG. 6 shows the generation process of an oligonucleotide stick during the implementation of the second aspect of the present invention according to an embodiment of the present invention. A method according to the second aspect of the present invention will be described with reference to FIGS. 5 and 6 as below:

Step (a): Selecting Start Position from Alignment Positions of Plurality of Target Nucleic Acid Sequences (210)

First, a start position is selected from alignment positions of a plurality of target nucleic acid sequences.

Unless especially stated otherwise herein, the step (a) in the second aspect of the present invention may be described with reference to the descriptions of step (a) in the first aspect of the present invention.

Since the descriptions of “target nucleic acid molecule”, “target molecule”, “target nucleic acid”, “target nucleic acid sequence”, “target sequence”, “a plurality of target nucleic acid sequences”, “alignment”, and “alignment positions” in the second aspect of the present invention are the same as those in the first aspect of the present invention, the common descriptions between them are omitted in order to avoid undue redundancy leading to the complexity of this specification.

In FIG. 6, the plurality of target nucleic acid sequences are sequences 1 to 5, and alignment positions expressed as serial numbers can be confirmed in the upper part of the alignment results in FIG. 6.

According to the present invention, a plurality of target nucleic acid sequences are aligned, and a start position is selected from alignment positions.

The start position in the present invention is any one selected from the alignment positions of the plurality of target nucleic acid sequences, and specifically, the start position is a conservative position or a non-conservative position of the alignment positions, more specifically, the start position is a conservative position, and most specifically, the start position is the first conservative position of the alignment positions. For example, the position of alignment no. 1 is a start position in the first round of stick generation in FIG. 6.

The alignment positions in the present invention include conservative and non-conservative positions of nucleotides of the plurality of target nucleic acid sequences aligned. However, the conservativity and non-conservativity are more strictly applied to the second aspect of the present invention compared with the first aspect of the present invention. Specifically, the conservativity in the second aspect refers to a case where the ratio of the number of a certain type of bases to the total number of bases is 100% at each alignment position, that is, a case where no different base exists at the alignment position, and the non-conservativity refers to a case where at least one different base exists at each alignment position.

In FIG. 6, the positions of alignment nos. 12, 22, and 24 are non-conservative positions, and the positions except for the non-conservative positions and a gap-containing position (the position of alignment no. 34) are conservative positions.

### Step (b): Selecting as End Position a Position Having the Minimum Number of Sequence Patterns (220)

Then, a position having the minimum number of sequence patterns within the predetermined allowable number of sequence patterns from the positions located a predetermined length or more apart from the start position is selected as an end position. The number of sequence patterns is determined by a plurality of target nucleic acid sequences that are aligned.

An end position selected in the present invention is any one of alignment positions of the plurality of target nucleic acid sequences which becomes an end point of a region constituting an oligonucleotide stick generated in the present invention and a position having the minimum number of sequence patterns within the predetermined allowable number of sequence patterns from the positions located a predetermined length or more apart from the start position is selected as an end position. That is, a position to be selected as an end position should satisfy the criterion regarding a length and is the criterion regarding the number of sequence patterns.

As for the predetermined length (minimum length) as a criterion regarding a length for selecting an end position, the predetermined length may be selected considering the length of an oligonucleotide to be designed from a designable region determined on the basis of oligonucleotide sticks. The predetermined length may be specifically 5, 10, 15, 20, 25, 30, or 35 nucleotides, but is not limited thereto. For example, the predetermined length may be one length selected from 5 to 100 nucleotides. The predetermined length is specifically 20, 25, or 30 nucleotides, more specifically 20 or 25 nucleotides, and most specifically 20 nucleotides. A criterion regarding a maximum value of the predetermined length is not particularly required. Since a designable region of oligonucleotides is determined from alignment positions of a plurality of target nucleic acid sequences on the basis of oligonucleotide sticks, a longer oligonucleotide stick is preferable as long as the oligonucleotide sticks satisfies generation criteria regarding a minimum length and the number of sequence patterns.

Herein, the number of positions may be exchangeably used with the number of bases. The term “nucleotide” used herein with referring to the number of positions, the number of bases, and lengths, may be exchangeably used with “base” or “mer”.

In FIG. 6, when the predetermined length of an oligonucleotide stick, as a criterion regarding a length, is 20 nucleotides, an oligonucleotide stick containing one sequence pattern in the first round of stick generation and oligonucleotide sticks containing one and two sequence patterns in the second round of stick generation fail to satisfy the criterion regarding the minimum length, and thus are treated as “dropouts”.

The criterion regarding the number of sequence patterns for selecting an end position is to have the minimum number of sequence patterns within the predetermined allowable number of sequence patterns among positions is satisfying the criterion regarding a minimum length. The predetermined allowable number of sequence patterns is selected from 5 to 60, but is not limited thereto. The predetermined allowable number of sequence patterns is selected from specifically 10 to 50, more specifically 10 to 40, and most specifically 20 to 30.

According to the present invention, the number of sequence patterns is determined by the plurality of target nucleic acid sequences that are aligned. Specifically, the number of sequence patterns is determined by grouping according to sequence identity of the plurality of target nucleic acid sequences that are aligned.

For example, referring to the position of alignment no. 1 to the position of alignment no. 26 in FIG. 6, the position of alignment no. 1 to the position of alignment no. 11 represent one sequence pattern since all the sequences are identical; the position of alignment no. 1 to the position of alignment no. 21 represent a total of two sequence patterns by having one sequence pattern of the sequence 2 and one sequence pattern grouping the sequences 1 and 3 to 5 since the sequence 2 has a different base, A base, from the other sequences at the position of alignment no. 12; the position of alignment no. 1 to the position of alignment no. 23 represent a total of three sequence patterns by having one sequence pattern of the sequence 2, one sequence pattern of the sequence 3, and one sequence pattern grouping the sequences 1, 4, and 5 since the sequence 3 has a different base, A base, from the other sequences at the position of alignment no. 22; and the position of alignment no. 1 to the position of alignment no. 26 represent a total of four sequence patterns by having one sequence pattern of the sequence 1, one sequence pattern of the sequence 2, one sequence pattern of the sequence 3, and one sequence pattern grouping the sequences 4 and 5 since the sequences 4 and 5 have a different base, A base, from the other sequences at the position of alignment no. 24.

In an embodiment, when the ratio of the number of sequence patterns to the total number of sequences is less than a predetermined ratio, such sequence patterns may not be considered in determining the number of sequence patterns. For example, the sequence pattern accounting for 1% or less of the total number of sequences is not considered.

According to an embodiment, the end position is a conservative position or a non-conservative position in the alignment positions, and more specifically, the end position is a conservative position.

According to an embodiment of the present invention, the end position in step (b) is present in two or more.

In cases where the predetermined length as a criterion regarding a length for selecting an end position is 20 nucleotides and the predetermined allowable number of sequence patterns as a criterion regarding the number of sequence patterns is 25, an end position is described with reference to FIG. 6 as below: When the position of alignment no. 1 is selected as a start position in the first round of stick generation in FIG. 6, the positions located 20 nucleotides or more apart from the position of alignment no. 1, that is, the positions satisfying the criterion regarding a minimum length are positions after the position of alignment no. 20. Out of the positions, an end position should be a position satisfying a minimum number of sequence patterns within 25 sequence patterns, that is, an end position should satisfy the criterion regarding the number of sequence patterns. It can be seen from FIG. 6 that the positions of alignment nos. 12, 22, and 24 have different bases and thus are non-conservative positions and the number of sequence patterns increases from the positions of alignment nos. 12, 22, and 24. Specifically, the number of sequence patterns increases from one to two at the position of alignment no. 12; the number of sequence patterns increases from two to three at the position of alignment no. 22; and the number of sequence patterns increases from three to four at the position of alignment no. 24. In such a case, the is positions having the minimum number of sequence patterns from the position of alignment no. 20 satisfying the criterion regarding a minimum length are the positions of alignment nos. 20 and 21 having two sequence patterns. Therefore, the positions of alignment nos. 20 and 21 may be selected as end positions satisfying the criterion regarding a minimum length and the criterion regarding the number of sequence patterns from the position of alignment no. 1.

According to an embodiment of the present invention, the alignment positions comprise a sequence pattern change position which is non-conservative position and at which the number of sequence pattern increases, and the end position in step (b) is selected from positions immediately before the sequence pattern change position. Specifically, the positions immediately before the sequence pattern change position are conservative positions immediately before the sequence pattern change position.

As described above, in FIG. 6, the positions of alignment nos. 12, 22, and 24 are all non-conservative positions and positions at which the number of sequence patterns increases, and therefore, the positions of alignment nos. 12, 22, and 24 are sequence pattern change positions (P). For example, suppose that the position of alignment no. 23 of the sequence 3 is not G base but A base in FIG. 6. Then, the position of alignment no. 23 is a non-conservative position but not a sequence pattern change position. The reason is that the number of sequence patterns increases from two to three at the position of alignment no. 22 while the position of alignment no. 23 is a non-conservative position but shows no increase in the number of sequence patterns (three). In addition, the positions of alignment nos. 25 and 26 are also non-conservative positions but are the same as the position of alignment no. 24 in view of the number of sequence patterns (four), so the positions of alignment nos. 25 and 26 are not sequence pattern change positions.

In the first round of stick generation in FIG. 6, the positions satisfying is the foregoing selection criterion for an end position are the positions of alignment nos. 20 and 21 and the sequence pattern change position is the position of alignment no. 22, so the position of alignment no. 21 which is the position (specifically the conservative position) immediately before the sequence pattern change position, may be selected as an end position.

### Step (c): Generating Oligonucleotide Stick (230)

Then, an oligonucleotide stick composed of a region from the start position to the end position is generated. The oligonucleotide stick comprises the number of sequence patterns and sequence pattern information determined by a plurality of target nucleic acid sequences that are aligned in the region

The term “generation or creation” used herein with referring to the oligonucleotide stick, does not mean the generation of a material oligonucleotide stick but the generation of sequence information of an oligonucleotide stick.

In the present invention, the region of the oligonucleotide stick contains the number of sequence pattern and sequence pattern information determined by a plurality of target nucleic acid sequences that are aligned in the region, and the sequence pattern information contains specifically conservative and non-conservative positions, sequence pattern change positions, types of conservative and non-conservative bases, and sequence information grouped into sequence patterns.

According to an embodiment of the present invention, the oligonucleotide stick in step (c) is a plurality of oligonucleotide sticks that have the same start position and the same number of sequence patterns but different end positions.

As described above, in FIG. 6, the positions of alignment nos. 20 and 21 are selected as end positions satisfying the criterion regarding a minimum length (20 nucleotides) and the criterion regarding the number of sequence patterns (the allowable number of sequence patterns: 25, the minimum number of sequence patterns: 2) for selecting an end position. In this case, oligonucleotide stick A composed of a region from the position of alignment no. 1, which is a start position, to the position of alignment no. 20, which is an end position having two sequence patterns from the start position, and oligonucleotide stick B composed of a region from the position of alignment no. 1, which is a start position, to the position of alignment no. 21, which is an end position having two sequence patterns from the start position, may be created, respectively.

According to an embodiment of the present invention, the oligonucleotide stick in step (c) is the longest oligonucleotide stick of oligonucleotide sticks having the same number of sequence patterns.

As described above, the oligonucleotide sticks A and B having different end positions may be created, but according to the present embodiment, from these, the oligonucleotide stick B having the longest length may be generated.

### Step (d): Repeating Generation of Oligonucleotide Stick (240)

Then, the generation of an oligonucleotide stick is repeated by selecting at least one start position different from the start position in step (a).

According to the present invention, a designable region of oligonucleotides covering a plurality of target nucleic acid sequences is determined from the plurality of target nucleic acid sequences, and thus it is necessary to create a plurality of oligonucleotide sticks containing information about a minimum number of sequence patterns from the positions located a predetermined length or more apart from different start position. Therefore, a procedure of selecting at least one start position different from the start position in step (a), selecting as an end position a position having the minimum number of sequence patterns within the predetermined allowable is number of sequence patterns from the positions located a predetermined length or more apart from the at least one different start position and then creating an oligonucleotide stick composed of a region from the at least one start position to the end position is repeated.

According to an embodiment of the present invention, in step (d), the at least one start position different from the start position in step (a) is selected from positions after non-conservative positions existing after the start position in step (a), specifically, selected from positions right after non-conservative positions existing after the start position in step (a), and more specifically, selected from conservative positions right after non-conservative positions existing after the start position in step (a).

According to an embodiment, in step (d), the at least one start position different from the start position in step (a) is sequentially selected from positions after non-conservative positions existing after the start position in step (a), specifically, sequentially selected from positions right after non-conservative positions existing after the start position in step (a), and more specifically, sequentially selected from conservative positions right after non-conservative positions existing after the start position in step (a).

For example, in FIG. 6, the first start position of an oligonucleotide stick in the first round of stick generation is the position of alignment no. 1, and the start position of an oligonucleotide stick created in the next second round of stick generation may be at least one selected from conservative positions (i.e., the positions of alignment nos. 13, 23, and 27) immediately after the non-conservative positions (i.e., the positions of alignment nos. 12, 22, and 24) existing after the first start position of the first round of stick generation, or alternatively, may be sequentially selected from the positions of alignment nos. 13, 23, and 27.

In an embodiment, when the start position is sequentially selected from the positions of alignment nos. 13, 23, and 27, as for the second round of stick generation in FIG. 6, the position of alignment no. 13, which is the conservative position right after the position of alignment no. 12, which is a non-conservative position existing after the position of alignment no. 1, which is the start position in the first round of stick generation, is used as a start position, and a position having the minimum number of sequence patterns (four) within 25 sequence patterns is selected as an end position from the positions located 20 nucleotides or more apart from the position of alignment no. 13, so a secondary oligonucleotide stick composed of a region from the start position to the end position is created. In such a manner, tertiary and quaternary oligonucleotide sticks are sequentially generated by using the positions of alignment nos. 23 and 27 as start positions for the third and fourth rounds of stick generation, respectively.

In FIG. 6, an oligonucleotide stick having four sequence patterns, created in the second round of stick generation, is an oligonucleotide stick composed of a region from the position of alignment no. 13 as a start position to the position of alignment no. 33 as an end position. Out of the plurality of target nucleic acid sequences that are aligned, the sequence 5 is a partial sequence including miss portions from the position of alignment no. 27 to the position of alignment no. 35 which is the 3′-end of the sequence 5. In such a case, the standard of whether the sequence pattern of the sequence 5 is considered is as follows: The standard may also be applied in the same manner when the sequence 5 is not a partial sequence but a gap-containing sequence in which there are as many gaps as miss portions. Specifically, when the length of the other portions excluding miss portions (or gaps) of a partial sequence (or a gap-containing sequence) in a region of an oligonucleotide stick to be created is a predetermined length or more, the other portions are considered as a sequence pattern, but the other portions are not considered as a sequence pattern when the length thereof is less than the predetermined length. For example, suppose that the predetermined length is 13 mers. Then, is the sequence 5 has a length of 14 mers and thus is considered as a separate sequence pattern. Therefore, the number of sequence patterns at the positions of alignment nos. 13 to 33 is a total of four by adding one sequence pattern grouping the sequences 1 and 2, one sequence pattern of the sequence 3, one sequence pattern of the sequence 4 and one sequence pattern of the sequence 5. However, for example, suppose that the positions of alignment nos. 25 and 26 of the sequence 5 are also miss portions. Then, the sequence 5 at the positions of alignment nos. 13 to 33 has a length of 12 mers, and thus the sequence 5 is not considered as a separate sequence pattern. Therefore, the number of sequence patterns at the positions of alignment nos. 13 to 33 is a total of three by adding one sequence pattern grouping the sequences 1 and 2, one sequence pattern of the sequence 3 and one sequence pattern of the sequence 4.

According to an embodiment of the present invention, the oligonucleotide sticks are generated or selected to satisfy at least one (specifically at least two, more specifically at least three, and still more specifically four) of the following criteria:

(i) a gap ratio; wherein when the alignment positions of the plurality of target nucleic acid sequences comprise a gap-containing position, the oligonucleotide sticks are generated by selecting as an end position a position before a gap-containing position having a gap ratio exceeding a predetermined gap ratio, and wherein the gap ratio represents a ratio between the number of gaps and the total number of bases at the gap-containing position and the total number of bases represents the sum of the numbers of existing bases and gaps,

(ii) a base exist ratio (BER) at each position of an oligonucleotide stick; wherein the BER represents a ratio between the sum of the numbers of existing bases and gaps at an alignment position corresponding to each position of an oligonucleotide stick and the total number of sequences that are aligned, and wherein the oligonucleotide stick is selected according to the number of positions each having a BER of less than a predetermined value,

(iii) a GC content; wherein a portion satisfying a predetermined GC content in an oligonucleotide stick is selected, and

(iv) amplicon region formation; wherein an amplicon region corresponding to a predetermined length in the 3′ direction from the 5′-end or in the 5′-direction from the 3′-end of an oligonucleotide stick is set, and oligonucleotide sticks included in the amplicon region are selected considering criteria regarding a stick base sum (SBS) and/or respective lengths of the oligonucleotide sticks included in the amplicon region.

According to the present invention, the oligonucleotide sticks may be generated or selected on the basis of criteria (i) to (iv) above as generation or selection criteria, in addition to being created on the basis of a length thereof and the number of sequence patterns as criteria. Therefore, criteria (i) to (iv) above are both creation criteria and selection criteria. For example, when criteria (i) to (iv) are creation criteria, the oligonucleotide sticks according to the method of the present invention may be created to satisfy at least one of criteria (i) to (iv), in addition to the criteria regarding a length thereof and the number of sequence patterns, and when criteria (i) to (iv) are selection criteria, the oligonucleotide sticks according to the method of the present invention may be created to satisfy the criteria regarding a length thereof and the number of sequence patterns and then selected to satisfy at least one of criteria (i) to (iv). Alternatively, at least one of criteria (i) to (iv) may be a creation criterion, and the other criteria may be selection criteria. Specifically, criterion (i) of criteria (i) to (iv) may be a generation criterion, and criteria (ii) to (iv) may be selection criteria.

According to an embodiment of the present invention, the oligonucleotide sticks may be created to satisfy criterion (i).

According to an embodiment, the oligonucleotide sticks may be selected to satisfy at least one of criteria (ii) and (iii).

According to an embodiment of the present invention, the oligonucleotide sticks may be selected to satisfy criterion (iv).

According to an embodiment, the oligonucleotide sticks may be created to satisfy criterion (i), selected to satisfy at least one of criteria (ii) and (iii), and selected to satisfy criterion (iv).

The oligonucleotide sticks of the present invention may be created or selected to satisfy criterion (i) regarding a gap ratio.

Since the descriptions of the criterion regarding a “gap ratio” in the first aspect of the present invention are the same as those in the second aspect of the present invention, the common descriptions between them are omitted in order to avoid undue redundancy leading to the complexity of this specification.

Unless especially stated otherwise in the second aspect of the present invention, the descriptions of the criterion regarding a gap ratio referring to FIG. 2 in the first aspect of the present invention may be equally applied to FIG. 6.

In FIG. 6, the sequences 1 to 3 have a portion where a base is absent, that is, a gap, at the position of alignment no. 34 according to the homology in the aligning procedure of a plurality of sequences. The sequence 5 is a partial sequence having portions where bases are absent (miss portions) at the positions of alignment nos. 27 to 35 which is the 3′-end of the sequence 5.

In FIG. 6, gaps exist at the position of alignment no. 34 in the sequence of the sequences 1 to 3, and thus the gap ratio at the position is 75%, which is a ratio of the number of gaps in the sequences 1 to 3 to the sum of the number of gaps of the sequences 1, 2, and 3 and the number, of G base of the sequence 4. Since the gap ratio exceeds a predetermined value (50%), the position of alignment no. 34 is a gap-containing position (G). The number of miss portions of sequence 5 at the position of alignment no. 34 is not considered in the calculation of the gap ratio.

Suppose that the gap ratio is a creation criterion and the predetermined gap ratio is 50%. Then, in FIG. 6, an oligonucleotide stick containing four sequence patterns in the second round of stick generation is created by using as an end position the position of alignment no. 33, which is a position immediately before the gap-containing position.

Alternatively, suppose that the gap ratio is a selection criterion and the predetermined gap ratio is 50%. Then, in FIG. 6, an oligonucleotide stick containing four sequence patterns in the second round of stick generation is created by using the position of alignment no. 35 as an end position, but may be selected by using as an end position the position of alignment no. 33, which is a position immediately before the gap-containing position exceeding the predetermined gap ratio.

In FIG. 6, the position of alignment no. 35, which is a conservative position right after the position of alignment no. 34, a gap-containing position, may be also selected as a start position.

The oligonucleotide stick of the present invention may be created or selected to satisfy criterion (ii) regarding a base exist ratio (BER) at each position of the oligonucleotide stick.

Since the descriptions of the criterion regarding “base exist ratio (BER) at each position of oligonucleotide stick” in the first aspect of the present invention are the same as those in the second aspect of the present invention, the common descriptions between them are omitted in order to avoid undue redundancy leading to the complexity of this specification.

The descriptions of the criterion regarding a base exist ratio (BER) with reference to FIG. 2 in the first aspect of the present invention may be equally applied to FIG. 6 in the second aspect of the present invention.

The oligonucleotide stick of the present invention may be created or selected to satisfy criterion (iii) regarding a GC content.

Since the descriptions of the criterion regarding “GC content” in the first aspect of the present invention are the same as those in the second aspect of the present invention, the common descriptions between them are omitted in order to avoid undue redundancy leading to the complexity of this specification.

The descriptions of the criterion regarding a GC content with reference to FIG. 3 in the first aspect of the present invention may be equally applied to the second aspect of the present invention.

The oligonucleotide stick of the present invention may be created or selected to satisfy criterion (iv) regarding amplicon region formation.

Since the descriptions of the criterion regarding “amplicon region formation” in the first aspect of the present invention are the same as those in the second aspect of the present invention, the common descriptions between them are omitted in order to avoid undue redundancy leading to the complexity of this specification.

The descriptions of the criterion regarding amplicon region formation with reference to FIG. 4 in the first aspect of the present invention may be equally applied to the second aspect of the present invention.

According to an embodiment of the present invention, the generation or selection criteria may further include v) a match ratio of a predetermined value between an oligonucleotide stick and a nucleic acid sequence of a non-target nucleic acid molecule

Since the descriptions of the “non-target nucleic acid molecule”, “non-target nucleic acid sequence”, and “match” in the first aspect of the present invention are the same as those in the second aspect of the present invention, the common descriptions between them are omitted in order to avoid undue m redundancy leading to the complexity of this specification.

The oligonucleotide sticks of the present invention contain the number of sequence patterns and sequence pattern information determined by a plurality of target nucleic acid sequences that are aligned, and therefore, a comparison is made of whether an oligonucleotide stick having such sequence pattern number and sequence pattern information is matched to a nucleic acid sequence of a non-target nucleic acid molecule. When the match ratio is considered through matching comparison, the number of sequence patterns included in an oligonucleotide stick may be considered. Specifically, when all sequence patterns of an oligonucleotide stick have a match ratio of a predetermined value, such an oligonucleotide stick may be neither created nor selected, or when some number of sequence patterns (e.g., one, two, three, or four) of all sequence patterns of an oligonucleotide stick or some ratio of sequence patterns (e.g., 1%, 2%, 3%, or 4%) to all sequence patterns thereof have a match ratio of a predetermined value, such an oligonucleotide may be neither created nor selected.

The predetermined value of the match ratio may be selected from 50% to 100%. For example, when the predetermined value of the match ratio is 100%, that is, when all sequence patterns of an oligonucleotide stick having sequence pattern number and sequence pattern information and a nucleic acid sequence of a non-target nucleic acid molecule are analyzed to be 100% matched to each other, such an oligonucleotide stick is neither generated nor selected, and other oligonucleotide sticks having sequence patterns showing a match ratio of less than 100% are created or selected.

According to an embodiment, the creation or selection of an oligonucleotide stick may be determined considering amplicon region forming ability of the oligonucleotide stick as well as the match ratio between the oligonucleotide stick and a non-target nucleic acid sequence. For example, suppose that the match ratios between all sequence patterns of the oligonucleotide sticks included in amplicon regions and a nucleic acid sequence of a non-target nucleic acid sequence are analyzed. Then, when at least one sequence pattern of at least one oligonucleotide stick of the oligonucleotide sticks included in an amplicon region shows a match ratio of less than 100%, oligonucleotide sticks included in the amplicon region are created or selected, and when all sequence patterns of all oligonucleotide sticks included in an amplicon region show a match ratio of 100%, the oligonucleotide sticks included in the amplicon region are neither created nor selected.

According to an embodiment of the present invention, the oligonucleotide sticks are ranked according to at least one (specifically, at least two, and most specifically three) of the following priority items:

(i) a ratio of an average base exist ratio (BER) to the number of sequence patterns of an oligonucleotide stick; the larger the ratio, the higher the priority;

(ii) an oligonucleotide stick length; the larger the length, the higher the priority; and

(iii) the number of amplicon regions in which one oligonucleotide stick is included; the larger the number, the higher the priority.

The oligonucleotide sticks generated (or selected) in the present invention may be ranked according to the priority items.

The present embodiment may be implemented considering the degree of creation (or selection) of oligonucleotide sticks or may be implemented independently without considering the degree.

Since the descriptions of the criterion considering the degree of creation (or selection) of oligonucleotide sticks in the first aspect of the present invention are the same as those in the second aspect of the present invention, the common descriptions between them are omitted in order to avoid undue redundancy leading to the complexity of this specification.

The term “average base exist ratio (BER)” used herein, refers to an average value of base exist ratios (BER) at respective positions of an oligonucleotide stick.

In an embodiment, the oligonucleotide sticks may be given scores and ranked according to at least one (specifically, at least two, and most specifically three) of the priority items. For example, when given scores and ranked on the basis of priority item (i), an oligonucleotide stick is ranked so that the score and priority is high as the ratio of the number of bases of the oligonucleotide stick to the number of non-conservative bases of the oligonucleotide stick is large.

In an embodiment, when given scores and ranked according to at least two of the priority items, the sum of the scores of respective items is obtained, and the larger the sum, the higher the rank of the oligonucleotide stick.

In an embodiment, the scores may be given according to the priority items by using different weights of the scores for the priority items. For example, the scores may be given by increasing the weight in order of priority items (i), (ii), and (iii).

According to an embodiment of the present invention, the method between steps (d) and (e), further comprises arranging amplicon regions according to the sum of the numbers of bases of oligonucleotide sticks ranked in a predetermined ranking or more among the oligonucleotide sticks included in the amplicon regions; selecting amplicon regions ranked in a predetermined ranking or more among the arranged amplicon regions; and selecting oligonucleotide sticks included in the selected amplicon regions.

Since the descriptions of step for arranging the amplicon region, step for selecting the amplicon region, and step for selecting the oligonucleotide stick in the first aspect of the present invention are the same as those in the second aspect of the present invention, the common descriptions between them are omitted in order to avoid undue redundancy leading to the complexity of this specification.

The oligonucleotide sticks included in the amplicon regions selected in such a manner are selected, and may be used to determine a designable region of oligonucleotides.

### Step (e): Determining Designable Region of Oligonucleotide (250)

Last, regions in an alignment of the plurality of target nucleic acid sequences, which correspond to the regions of the oligonucleotide sticks, are determined as a designable region of oligonucleotides.

When the oligonucleotide sticks have no overlapping areas, respective areas of the oligonucleotide sticks correspond to a region of the oligonucleotide sticks, and when the oligonucleotide sticks have overlapping areas, a region linking the overlapping areas corresponds to a region of the oligonucleotide sticks.

Since the descriptions of the “oligonucleotide”, “primer”, and “probe” in the first aspect of the present invention are the same as those in the second aspect of the present invention, the common descriptions between them are omitted in order to avoid undue redundancy leading to the complexity of this specification.

According to an embodiment of the present invention, the designable region is a designable region of oligonucleotides that permits to exhibit a maximum target coverage for the plurality of target nucleic acid sequences with at least two primer pair and/or at least two probe.

According to an embodiment of the present invention, the at least two primer pairs and/or the at least two probes are expressed as the at least two oligonucleotide groups.

According to an embodiment, at least one primer or probe in the primers and probes included in a first oligonucleotide group is different from at least one primer or probe in the primers and probes included in a second oligonucleotide group. According to an embodiment, the primers or probes in the first and second oligonucleotide groups have different base sequences or configurations except for a base (e.g., a degenerate base) used for a maximum target coverage.

According to the present embodiment, when a designable region is determined on the basis of oligonucleotide sticks containing a position is satisfying the criterion regarding a minimum length and the criterion regarding the number of sequence patterns, it means that a plurality of target nucleic acid sequences to be amplified or detected by an oligonucleotide have sequence similarity enough to be covered by two or more primer pairs and/or two or more probes.

According to an embodiment of the present invention, the method of the present invention is performed by computer-implemented methods. A storage medium, a device, and a computer program for performing the above-described method of the present invention on a computer will be described in detail as below.

A method according to the first aspect of the present invention and a method according to the second aspect of the present invention may be implemented independently with each other. Alternatively, when a designable region of oligonucleotides is not determined by the method according to the first aspect, the method according to the second aspect may be consecutively carried out, and vice versa. When not determined by the above manner, a designable region of oligonucleotides may be determined by selecting another target nucleic acid molecule instead of a target nucleic acid molecule of interest (e.g., a target gene).

### Storage Medium, Device, Program

In another aspect of the present invention, there is provided a computer readable storage medium containing instructions to configure a processor to perform a method for determining a designable region of oligonucleotides in a plurality of target nucleic acid sequences having sequence similarity, the method comprising: (a) selecting a start position from alignment positions of a plurality of target nucleic acid sequences; (b) selecting as an end position a position having the minimum number of sequence patterns within the predetermined allowable number of sequence patterns from the positions located a predetermined length or more apart from the start position; wherein the number of sequence patterns is determined by a plurality of target nucleic acid sequences that are aligned, (c) generating an oligonucleotide stick composed of a region from the start position to the end position, wherein the oligonucleotide stick comprises the number of sequence patterns and sequence pattern information determined by a plurality of target nucleic acid sequences that are aligned in the region; (d) repeating the generation of an oligonucleotide stick by selecting at least one start position different from the start position in step (a); and (e) determining as a designable region of oligonucleotides regions in an alignment of the plurality of target nucleic acid sequences, which correspond to the regions of the oligonucleotide sticks.

In still another aspect of the present invention, there is provided a computer program to be stored on a computer readable storage medium, to configure a processor to perform a method for determining a designable region of oligonucleotides in a plurality of target nucleic acid sequences having sequence similarity, the method comprising: (a) selecting a start position from alignment positions of a plurality of target nucleic acid sequences; (b) selecting as an end position a position having the minimum number of sequence patterns within the predetermined allowable number of sequence patterns from the positions located a predetermined length or more apart from the start position; wherein the number of sequence patterns is determined by a plurality of target nucleic acid sequences that are aligned, (c) generating an oligonucleotide stick composed of a region from the start position to the end position, wherein the oligonucleotide stick comprises the number of sequence patterns and sequence pattern information determined by a plurality of target nucleic acid sequences that are aligned in the region; (d) repeating the generation of an oligonucleotide stick by selecting at least one start position different from the start position in step (a); and (e) determining as a designable region of oligonucleotides regions in an alignment of the plurality of is target nucleic acid sequences, which correspond to the regions of the oligonucleotide sticks.

In another aspect of the present invention, there is provided a device for determining a designable region of oligonucleotides in a plurality of target nucleic acid sequences having sequence similarity, comprising (a) a computer processor, and (b) a computer readable storage medium of the present method coupled to the computer processor.

Since the storage medium, the device, and the computer program of the present invention are intended to perform the present methods described hereinabove in a computer, the common descriptions between them are omitted in order to avoid undue redundancy leading to the complexity of this specification.

The program instructions are operative, when performed by the processor, to cause the processor to perform the method of the present invention described above. The program instructions for performing a method for determining a designable region of oligonucleotides may comprise the following instructions: (i) an instruction to select a start position from alignment positions of a plurality of target nucleic acid sequences; (ii) an instruction to select as an end position a position having the minimum number of sequence patterns within the predetermined allowable number of sequence patterns from the positions located a predetermined length or more apart from the start position; (iii) an instruction to generate an oligonucleotide stick composed of a region from the start position to the end position; (iv) an instruction to repeat the generation of an oligonucleotide stick by selecting at least one start position different from the start position in instruction (i); and (v) an instruction to determine (e.g., display on an out device) as a designable region of oligonucleotides regions in an alignment of the plurality of target nucleic acid sequences, which correspond to the regions of the oligonucleotide sticks.

Since the descriptions of the processor, the type of computer readable storage medium, the manner in which a designable region is provided, the instructions to configure the processor that may be included in a logic system, and the computer processor in the first aspect of the present invention are the same as those in the second aspect of the present invention, the common descriptions between them are omitted in order to avoid undue redundancy leading to the complexity of this specification.

The features and advantages of this invention are summarized as follows.

(a) In determining a designable region of oligonucleotides for detecting a plurality of nucleic acid sequences of a target nucleic acid molecule exhibiting genetic diversity with a maximum target coverage, the present invention provides a more logical and efficient method by adopting a strategy of generating oligonucleotide sticks having sequence information about non-conservative positions within a predetermined allowable number or a minimum number of sequence patterns within a predetermined allowable number of sequence patterns while having a predetermined length or more from the alignment results of a plurality of target nucleic acid sequences, thereby providing a designable region of oligonucleotides.

(b) According to the present invention, both a target coverage and method for designing an oligonucleotide are considered in determining a designable region of oligonucleotides. Specifically, according to the present invention, with respect to the target coverage, a designable region of oligonucleotides with a maximum target coverage can be determined by applying generation criteria for including a predetermined allowable number of conservative positions or creation criteria having a minimum number of sequence patterns within a predetermined allowable number of sequence patterns to a plurality of nucleic acid sequences of a target nucleic acid molecule.

Furthermore, according to an embodiment of the present invention, with respect to a method for designing the oligonucleotide, one oligonucleotide (one primer pair and/or one probe) can be designed in a designable region determined on the basis of generation criteria considering the number of conservative positions, and a combination of two or more oligonucleotides (two or more primer pairs and/or two or more probes) can be designed in a designable region determined on the basis of generation criteria considering the number of sequence patterns.

(c) When a designable region (specifically, a conservative region) of oligonucleotides for detecting a plurality of nucleic acid sequences of a target nucleic acid molecule exhibiting genetic diversity with a maximum target coverage is selected, the conventional methods were an empirical and manually selected methods, which are time-consuming and labor-consuming methods with poor speed and accuracy.

According to the present invention, a designable region of oligonucleotides can be determined in a logical and automatic manner unlike the conventional methods described above, and the methods of the present invention are more speedy and accurate than any other conventional method. Furthermore, the present invention can provide a variety of regions as a designable region, and particularly, can provide a region that has not been selected in the conventional method, that is, a region that may be missed as a designable region.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1: Selecting Designable Region of Oligonucleotide

### Alignment of Target Nucleic Acid Sequences

Hemagglutinin-neuraminidase (HN) gene sequences of human parainfluenza virus type 2 (PIV2), as a plurality of target nucleic acid sequences, collected from the National Center for Biotechnology Information (NCBI), were aligned. The alignment results were obtained having the positions of alignment nos. 1 to 2230, that is, 2230 base positions.

In the present example, the alignment results were analyzed in a single stick manner, which corresponds to a first aspect of the present invention, to investigate regions that can be amplified and detected with a combination of one pair of primers and one probe.

### Generation of Single Sticks

Single sticks were generated from an alignment of HN gene sequences of PIV2. Sticks were generated containing up to two (i.e., 0, 1, and 2) variation positions (non-conservative positions) from the conservative position (the start position) of the first position of the alignment positions, end positions were set to the positions immediately before the first, second, and third variation positions existing from the start position such that the longest sticks can be generated according to the number of variation positions that are contained in the sticks. In addition, second round single sticks from a conservative position immediately after the first variation position from the start position were generated in the same manner. Subsequently, in such a manner, sticks were created on the alignment while changing the start position.

Out of the alignment positions of the plurality of target nucleic acid sequences, a position at which the ratio of the number of a different type of bases to the total number of bases is more than 1% (a case where the total number of aligned sequences is less than 3000) or the total number of a different type of bases is more than 30 (a case where the total number of aligned sequences is 3000 or more) was defined as a variation position. A complex base, such as R or Y, was also determined as a different base. Separately from the variation positions, a position at which a gap exists (a gap-containing position) was defined. A position at which the ratio of the number of gaps to the total number of bases is more than 1% (a case where the total number of aligned sequences is less than 3000) or the total number of gaps is more than 30 (a case where the total number of aligned sequences is 3000 or more) was also defined as the gap-containing position.

The gap is inevitably inserted during the alignment of sequences and represents a portion at which a base is absent in a sequence, and the gap was distinguished from a miss portion of a partial sequence. The length of the sticks is at least 20 mers and the sticks contained no gap. Therefore, a stick can be extended only up to a position right before a gap-containing position, and a new stick is created from a position immediately after a gap-containing position.

Through such a manner, approximately 237 single sticks were created to the last of the alignment positions, and ten out of these single sticks are shown as examples in Table 1.

TABLE 1

| Start position | End position | Length | Number of variation positions |
|---|---|---|---|
| 1 | 39 | 39 | 1 |
| 11 | 39 | 29 | 0 |
| 41 | 64 | 24 | 1 |
| 66 | 132 | 67 | 2 |
| 67 | 132 | 66 | 1 |
| 68 | 132 | 65 | 0 |
| 134 | 156 | 23 | 2 |
| 136 | 156 | 21 | 1 |
| 243 | 264 | 22 | 2 |
| 245 | 264 | 20 | 1 |

In Table 1, the start and end positions represent alignment positions of a plurality of HN gene sequences of PIV2.

### Basic Filter

The generated sticks were selected to satisfy the basic filter criteria as below: (i) When the base exist ratio (BER) was calculated at each base position of the sticks, sticks having more than 10 base positions, at which the base exist ratio (BER) was less than 30%, were excluded. Here, the BER represents a ratio of the sum of the numbers of existing bases and gaps at the alignment position of a plurality of target nucleic acid sequences, corresponding to each position of single sticks, to the total number of aligned sequences. (ii) While the 20-mer unit was shifted on each of the generated single sticks, a portion of the single stick, at which the content of GC existing relative to 20 bases (20-mer) was 20% (4 mers) or less, was excluded.

### Investigation of Amplicon Forming Ability (Amplicon Filter)

Since a primer pair should form an amplicon when a primer and/or a probe are designed from a designable region, sticks capable of forming an amplicon by forming a combination of generated single sticks were selected. Specifically, a region from the 5′-end to 350 bases for each stick was designated as a 350-base amplicon region according to the standard that the maximum length of an amplicon does not exceed 350 bases. Out of the single sticks passing through the basic filter, single sticks, which are included in a 350-base amplicon region to combine with two or more sticks and in which the stick base sum (SBS) of the sticks included in the amplicon region is 150 bases or more and at least one stick included in the amplicon region is 100 bases or more in length or the number of sticks having a length of 40 bases or more is two or more, were selected.

In the calculation of the SBS, the number of overlapping bases between the sticks was considered only once. For example, when two sticks included in is one amplicon region are 50 bases in length and have 10 overlapping bases, the SBS is 90 bases. In such a manner, a total of 172 single sticks passing through the basic filter and the amplicon filter were selected, and 10 out of the single sticks are shown as examples in Table 2 below.

TABLE 2

| Start position | End position | Length | Number of variation positions |
|---|---|---|---|
| 266 | 291 | 26 | 2 |
| 267 | 311 | 45 | 2 |
| 267 | 291 | 25 | 1 |
| 273 | 311 | 39 | 1 |
| 273 | 339 | 67 | 2 |
| 293 | 339 | 47 | 1 |
| 293 | 342 | 50 | 2 |
| 313 | 339 | 27 | 0 |
| 313 | 342 | 30 | 1 |
| 313 | 356 | 44 | 2 |

In Table 2, the start and end positions represent alignment positions of a plurality of HN gene sequences of PIV2.

### Investigation of Degree of Generation of Single Sticks (Stick and Amplicon Alignment) and Determination of Designable Region

The degree of generation of sticks was investigated by finding the stick base sum (SBS) of all the sticks selected through the basic filter and the amplicon filter. In the calculation of the SBS, the number of overlapping bases between sticks was considered only once. Sticks satisfying at least one of the following standards were determined to have an appropriate length to design a primer and/or a probe, and a region of the alignment corresponding to the sticks was determined as a designable region: i) the stick base sum (SBS) of all the selected sticks is less than 600 bases (absolute standard), and ii) the ratio of the SBS to the number of alignment positions having a BER of 30% or more among alignment positions of a plurality of target nucleic acid sequences is less than 60% (relative standard)

When sticks do not satisfy all the standards, that is, the stick base sum of the selected sticks is larger than absolute and relative standards, additional alignment and selection procedures may be carried out as follows. Above all, a single stick was given scores and ranked on the basis of the following priority items: First, a higher score was given as the ratio of the number of stick bases to the number of variation positions (non-conservative positions) in the single stick is large. Second, a higher score was given as the average base exist ratio (BER) of the single stick is large. Third, a higher score was given as the number of amplicon regions in which one oligonucleotide stick is included is large.

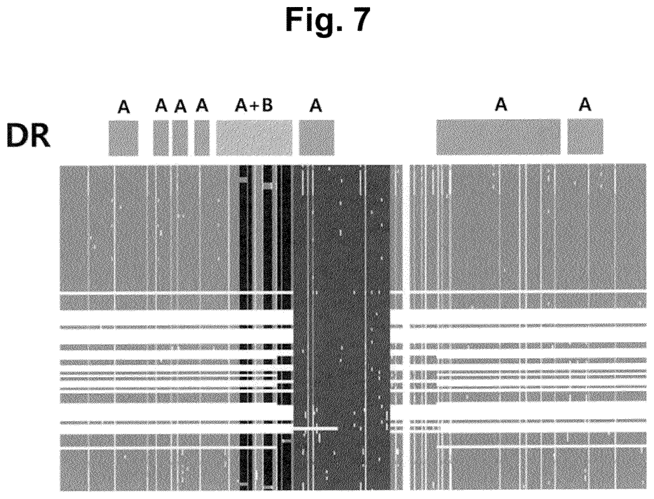

Then, the sum of the scores according to the above three standards was obtained and the sticks were arranged in descending order of sum, and the top 30% single sticks were selected. Then, the sum of the number of bases of the sticks corresponding to the top 30% single sticks of the single sticks included in the amplicon region was obtained; amplicon regions were arranged in descending order of sum; the top 50% amplicon regions were sequentially selected; and then the single sticks included in each amplicon region were linked, so a region in the alignments of the plurality of target nucleic acid sequences, which corresponds to the linked single sticks, was determined as a designable region. The results can be confirmed in Table 3 and FIG. 7 below.

### Comparison of Designable Region

A comparison was made between a designable region determined through the above method (Example 1) and a conventionally known designable region (Control 1) which is manually selected by the naked eye, and the results are shown in Table 3 and FIG. 7 below:

TABLE 3

| Example 1 | | | | | |
|---|---|---|---|---|---|
| Single stick | | | Control 1 | | |
| Start position | End position | Length | Start position | End position | Length |
| 466 | 560 | 95 | — | | |
| 574 | 596 | 23 | | | |
| 643 | 682 | 40 | | | |
| 689 | 726 | 38 | | | |
| 730 | 851 | 122 | 805 | 841 | 37 |
| 862 | 905 | 44 | 875 | 899 | 25 |
| 913 | 1002 | 90 | 918 | 956 | 39 |

| | | | |
|---|---|---|---|
| 1006 | 1034 | 29 | — |
| 1042 | 1143 | 102 | |
| 1442 | 1494 | 53 | |
| 1505 | 1653 | 149 | |
| 1655 | 1730 | 76 | |
| 1744 | 1830 | 87 | |
| 1836 | 1908 | 73 | |
| 1927 | 1995 | 69 | |

It can be confirmed from Table 3 and FIG. 7 that the designable region determined in Example 1 included the conventionally known designable region which is manually selected by the naked eye. In FIG. 7, a portion indicated by A+B represents a designable region of Example 1 and a hybridization region of Control 1, and each of the other portions indicated by A represents a designable region determined in Example 1.

The determination of a designable region through Example 1 took 1.475 s, and the designable region in the 2230-mer sequence of the target gene corresponded to 1090 mers. That is, about 49% of the target gene was selected as a designable region. It was therefore confirmed that in order to allow one primer pair and/or one probe to cover a plurality of target nucleic acid sequences with sequence similarity, a region capable of designing the one primer pair and/or one probe can be determined with speed and accuracy.

### Example 2: Selection of Designable Region of Oligonucleotide

### Alignment of Target Nucleic Acid Sequences

F gene sequences of Human metapueumovirus (hPMV), as a plurality of target nucleic acid sequences, collected from the National Center for Biotechnology Information (NCBI), were aligned. The alignment results were obtained having the positions of alignment nos. 1 to 1699, that is, 1699 base positions.

In the same manner as in Example 1, single sticks were first generated, and then a designable region of an oligonucleotide was determined.

### Generation of Single Sticks, Basic Filter, and Investigation of Amplicon Forming Ability (Amplicon Filter)

Single sticks were created and selected through a basic filter by the same method as described in Example 1, but there was no stick passing through an amplicon filter. Therefore, through a pattern stick manner corresponding to a second aspect of the present invention, a region that can be amplified and detected with a combination of two or more primer pairs and/or two or more probes was investigated.

### Generation of Pattern Sticks

Pattern sticks were created from an alignment of F gene sequences of hPMV. As for the creation of the pattern sticks, a plurality of nucleic acid sequences are grouped from the first position to a position at which the number of sequence patterns increases according to the sequence identity, and the longest stick of sticks having a minimum number of sequence patterns within 25 sequence patterns while satisfying a length of at least 20 mers (at least 20 bases or at least 20 positions) from the first position was generated as a pattern stick. In addition, pattern sticks from the conservative position immediately after the first variation position from the first position were created in the same manner.

Subsequently, in such a manner, pattern sticks were generated on the alignment while changing the start position. In addition, 1% or less of sequence patterns to the total number of aligned sequences were ignored. The sticks were also set to contain no gap. Specifically, when the alignment is positions of the plurality of F gene sequences of hPMV include at least one gap-containing position and the number of gaps to the total number of bases (including the number of gaps) exceeds 50% at the gap-containing position, a stick was set to be created from a start position to the position right before the gap-containing position. That is, a stick can be extended only to a position immediately before a gap-containing position, and a new stick is created from a position right after the gap-containing position.

Here, the gap represents a portion where a base is absent in the aligned sequences, and was distinguished from a region having no sequence information, such as a sequence registered as a partial sequence. However, a pattern stick was created at a portion other than a gap-containing position when the gap ratio was more than 99%, and a pattern containing a gap-containing position with a gap ratio of 99% or more was disregarded due to a sequence pattern of 1% or less.

Meanwhile, the pattern sticks are created to determine a region for being capable of designing two or more primer pairs and/or two or more probes, and thus may contain a plurality of gaps compared with single sticks. In such a case, the number of patterns may be calculated as follows. Specifically, suppose that a sequence containing gaps and a sequence containing no gap are included in the sequence information contained in the generated stick. Then, the number of sequence patterns can be calculated when the number of bases existing except for gaps is 13 mers or more in the sequence containing a gap. The sequence containing gaps is not considered in the calculating of the number of sequence patterns when the number of bases existing except for gaps is less than 13 mers in the sequence containing gaps.

In such a case, the number of sequence patterns is calculated by only the sequence containing no gap. Such the calculation of the number of pattern can also be equally applied to a partial sequence.

Through such a manner, approximately 1232 pattern sticks were generated to the last of the alignment positions, and ten out of these pattern sticks are shown as examples in Table 4.

TABLE 4

| Start position | End position | Length |
|---|---|---|
| 19 | 38 | 20 |
| 24 | 44 | 21 |
| 25 | 44 | 20 |
| 27 | 47 | 21 |
| 28 | 47 | 20 |
| 29 | 48 | 20 |
| 33 | 53 | 21 |
| 35 | 57 | 23 |
| 37 | 57 | 21 |
| 38 | 57 | 20 |

In Table 4, the start and end positions represent alignment positions of a plurality of F gene sequences of hPMV.

### Basic Filter

The generated sticks were selected to satisfy the basic filter criteria as below: (i) When the base exist ratio (BER) was calculated at each base position of the sticks, sticks having more than 10 base positions, at which the base exist ratio (BER) was less than 30%, were excluded. Here, the BER represents a ratio of the sum of the numbers of existing bases and gaps at the alignment position of a plurality of target nucleic acid sequences, corresponding to each position of pattern sticks, to the total number of aligned sequences. (ii) While the 20-mer unit was shifted on each of the generated pattern sticks, a partial position of the pattern stick, at which the content of GC existing relative to 20 bases (20-mer) was 10% (2 mers) or less, was excluded.

### Investigation of Amp/Icon Forming Ability (Amplicon Filter)

In order to select sticks capable of forming an amplicon by forming a is combination of generated pattern sticks, the amplicon forming ability of pattern sticks was investigated by the same method as in Example 1 except that the stick base sum (SBS) of the sticks included in a 350-base amplicon region was 80 bases but not 150 bases according to the amplicon filter standard of a single stick. In such a manner, a total of 1141 pattern sticks passing through the basic filter and the amplicon filter were selected, and 10 out of the pattern sticks are shown as examples in Table 5 below.

TABLE 5

| Start position | End position | Length | Number of sequence patterns |
|---|---|---|---|
| 163 | 182 | 20 | 7 |
| 167 | 188 | 22 | 10 |
| 169 | 188 | 20 | 10 |
| 170 | 191 | 22 | 11 |
| 172 | 191 | 20 | 7 |
| 175 | 197 | 23 | 7 |
| 176 | 197 | 22 | 7 |
| 178 | 197 | 20 | 6 |
| 184 | 206 | 23 | 8 |
| 187 | 206 | 20 | 7 |

In Table 5, the start and end positions represent alignment positions of a plurality of F gene sequences of hPMV.

### Investigation of Degree of Generation of Pattern Sticks (Stick and Amplicon Alignment) and Determination of Designable Region

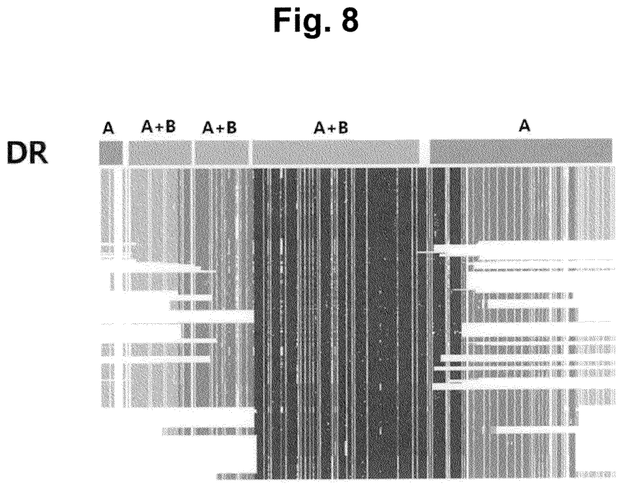

The same method was carried out as the investigation of the degree of generation of single sticks (stick and amplicon alignment) and the determination of a designable region in Example 1. However, the first and second standards of the standards for arrangement of sticks were as follows. First, a higher score was given as the ratio of average base exist ratio (BER) to the number of sequence pattern in a pattern stick is large. Second, a higher score was given as the length of the pattern stick is large. Then, the sum of the scores according to the above standards was obtained and the sticks were arranged in descending order, and the top 30% pattern sticks were selected. Then, the sum of the number of bases of the sticks corresponding to the top 30% patterns sticks of the pattern sticks included in the amplicon region was obtained; amplicon regions were arranged in descending order of sum; the top 50% amplicon regions were sequentially selected; and then the pattern sticks included in each amplicon region were linked, so a region in the alignments of the plurality of target nucleic acid sequences, which corresponds to the linked pattern sticks, was determined as a designable region. The results can be confirmed in Table 6 and FIG. 8 below.

### Comparison of Designable Region

A comparison was made between a designable region determined through the above method (Example 2) and a conventionally known designable region (Control 2) which is manually selected by the naked eye, and the results are shown in Table 6 and FIG. 8 below:

TABLE 6

| Example 2 | | | | | | |
|---|---|---|---|---|---|---|
| | Pattern stick | | | Control 2 | | |
| | Start | End | | Start | End | |
| Single stick | position | position | Length | position | position | Length |
| No amplicon- | 415 | 575 | 161 | — | | |
| forming stick | 580 | 607 | 28 | | | |
| | 611 | 641 | 31 | | | |
| | 667 | 748 | 82 | 671 | 704 | 34 |
| | 751 | 815 | 65 | 757 | 782 | 26 |
| | 818 | 1085 | 268 | 829 | 933 | 105 |
| | 1107 | 1135 | 29 | 984 | 1022 | 39 |

| | | | |
|---|---|---|---|
| 1137 | 1157 | 21 | — |
| 1159 | 1372 | 214 | |
| 1379 | 1505 | 127 | |

It can be confirmed from Table 6 and FIG. 8 that the designable region determined in Example 2 included the conventionally known designable region which is manually selected by the naked eye. In FIG. 8, a portion indicated by A+B represents a hybridization region of in Example 2 and a designable region in Control 1, and each of the other portions indicated by A represents a designable region determined in Example 2. The determination of a designable region through Example 2 took 23.75 s, and the designable region in the 1699-mer sequence of the target gene corresponded to 1090 mers. That is, about 60% of the target gene was selected as a designable region. It was therefore confirmed that in order to allow two or more primer pairs and/or two or more probes to cover a plurality of F gene sequences of hPMV, a region for being capable of designing the primer pairs and/or probes can be determined with speed and accuracy.

Having described a preferred embodiment of the present invention, it is to be understood that variants and modifications thereof falling within the spirit of the invention may become apparent to those skilled in this art, and the scope of this invention is to be determined by appended claims and their equivalents.

1. A method for determining a designable region of oligonucleotides in a plurality of target nucleic acid sequences having sequence similarity, comprising:
(a) selecting a start position from alignment positions of a plurality of target nucleic acid sequences; wherein the alignment positions comprise a conservative position and a non-conservative position of nucleotides of the plurality of target nucleic acid sequences that are aligned, the conservative position has one type of bases exhibiting conservativity, and the non-conservative position has two or more types of bases exhibiting non-conservativity;
(b) selecting as an end position a position comprising a non-conservative position within a predetermined allowable number from the start position;
(c) generating an oligonucleotide stick composed of a region from the start position to the end position; wherein the oligonucleotide stick comprises sequence information determined by a plurality of target nucleic acid sequences that are aligned in the region;
(d) repeating the generation of an oligonucleotide stick by selecting at least one start position different from the start position in step (a); and
(e) determining as a designable region of oligonucleotides the regions in an alignment of the plurality of target nucleic acid sequences, which correspond to the regions of the oligonucleotide sticks.

2. The method according to claim 1, wherein the end position in step (b) is present in two or more, and the oligonucleotide stick in step (c) is a plurality of oligonucleotide sticks that have the same start position and the same number of non-conservative positions but different end positions.

3. The method according to claim 1, wherein the oligonucleotide stick in step (c) is the longest oligonucleotide stick of oligonucleotide sticks comprising only conservative positions, or the longest oligonucleotide stick of oligonucleotide sticks having the same number of non-conservative positions.

4. The method according to claim 1, wherein in step (d), the at least one start position different from the start position in step (a) is selected from positions after non-conservative positions existing after the start position in step (a).

5. The method according to claim 1, wherein in step (d), the at least one start position different from the start position in step (a) is sequentially selected from positions after non-conservative positions existing after the start position in step (a).

6. The method according to claim 1, wherein the start and end positions are conservative positions.

7. (canceled)

8. The method according to claim 1, wherein the predetermined allowable number is 1, 2, 3, 4, or 5.

9. The method according to claim 1, wherein the oligonucleotide sticks are generated or selected to satisfy at least one of the following criteria:
(i) a predetermined minimum length of an oligonucleotide stick,
(ii) a gap ratio; wherein when the alignment positions of the plurality of target nucleic acid sequences comprise a gap-containing position, the oligonucleotide sticks are generated by selecting as an end position a position before a gap-containing position having a gap ratio exceeding a predetermined gap ratio, and wherein the gap ratio represents a ratio between the number of gaps and the total number of bases at the gap-containing position and the total number of bases represents the sum of the numbers of existing bases and gaps,
(iii) a base exist ratio (BER) at each position of an oligonucleotide stick; wherein the BER represents a ratio between the sum of the numbers of existing bases and gaps at an alignment position corresponding to each position of an oligonucleotide stick and the total number of sequences that are aligned, and wherein the oligonucleotide stick is selected according to the number of positions each having a BER of less than a predetermined value,
(iv) a GC content; wherein a portion satisfying a predetermined GC content in an oligonucleotide stick is selected, and
(v) amplicon region formation; wherein an amplicon region corresponding to a predetermined length in the 3′ direction from the 5′-end or in the 5′-direction from the 3′-end of an oligonucleotide stick is set, and oligonucleotide sticks included in the amplicon region are selected considering criteria regarding a stick base sum (SBS) and/or respective lengths of the oligonucleotide sticks included in the amplicon region.

10. (canceled)

11. The method according to claim 1, wherein the oligonucleotide sticks are ranked according to at least one of the following priority items:
(i) a ratio of the number of bases of an oligonucleotide stick to the number of non-conservative bases of the oligonucleotide stick; the larger the ratio, the higher the priority;
(ii) an average base exist ratio (BER) of an oligonucleotide stick; the larger the average BER, the higher the priority, and
(iii) the number of amplicon regions in which one oligonucleotide stick is included; the larger the number, the higher the priority.

12.-13. (canceled)

14. The method according to claim 1, wherein the designable region is a designable region of oligonucleotides that permits to exhibit a maximum target coverage for the plurality of target nucleic acid sequences with one primer pair and/or one probe.

15.-16. (canceled)

17. A computer readable storage medium containing instructions to configure a processor to perform a method for determining a designable region of oligonucleotides in a plurality of target nucleic acid sequences having sequence similarity, the method comprising:
(a) selecting a start position from alignment positions of a plurality of target nucleic acid sequences; wherein the alignment positions comprise a conservative position and a non-conservative position of nucleotides of the plurality of target nucleic acid sequences that are aligned, the conservative position has one type of bases exhibiting conservativity, and the non-conservative position has two or more types of bases exhibiting non-conservativity;
(b) selecting as an end position a position comprising a non-conservative position within a predetermined allowable number from the start position;
(c) generating an oligonucleotide stick composed of a region from the start position to the end position; wherein the oligonucleotide stick comprises sequence information determined by a plurality of target nucleic acid sequences that are aligned in the region;
(d) repeating the generation of an oligonucleotide stick by selecting at least one start position different from the start position in step (a); and
(e) determining as a designable region of oligonucleotides the regions in an alignment of the plurality of target nucleic acid sequences, which correspond to the regions of the oligonucleotide sticks.

18. A method for determining a designable region of oligonucleotides in a plurality of target nucleic acid sequences having sequence similarity, comprising:
(a) selecting a start position from alignment positions of a plurality of target nucleic acid sequences;
(b) selecting as an end position a position having the minimum number of sequence patterns within the predetermined allowable number of sequence patterns from the positions located a predetermined length or more apart from the start position; wherein the number of sequence patterns is determined by a plurality of target nucleic acid sequences that are aligned,
(c) generating an oligonucleotide stick composed of a region from the start position to the end position; wherein the oligonucleotide stick comprises the number of sequence patterns and sequence pattern information determined by a plurality of target nucleic acid sequences that are aligned in the region,
(d) repeating the generation of an oligonucleotide stick by selecting at least one start position different from the start position in step (a); and
(e) determining as a designable region of oligonucleotides the regions in an alignment of the plurality of target nucleic acid sequences, which correspond to the regions of the oligonucleotide sticks.

19. The method according to claim 18, wherein the end position in step (b) is present in two or more, and the oligonucleotide stick in step (c) is a plurality of oligonucleotide sticks that have the same start position and the same number of sequence patterns but different end positions.

20. The method according to claim 18, wherein the alignment positions comprise a sequence pattern change position which is non-conservative position and at which the number of sequence pattern increases, and the end position in step (b) is selected from positions immediately before the sequence pattern change position.

21. The method according to claim 18, wherein the oligonucleotide stick in step (c) is the longest oligonucleotide stick of oligonucleotide sticks having the same number of sequence patterns.

22. The method according to claim 18, wherein in step (d), the at least one start position different from the start position in step (a) is selected from positions after non-conservative positions existing after the start position in step (a).

23. The method according to claim 18, wherein in step (d), the at least one start position different from the start position in step (a) is sequentially selected from positions after non-conservative positions existing after the start position in step (a).

24. The method according to claim 18, wherein the start and end positions are conservative positions.

25. The method according to claim 18, wherein the predetermined allowable number of sequence patterns is selected from 10 to 40.

26. The method according to claim 18, wherein the predetermined length is 20 nucleotides.

27. The method according to claim 18, wherein the oligonucleotide sticks are generated or selected to satisfy at least one of the following criteria:
(i) a gap ratio; wherein when the alignment positions of the plurality of target nucleic acid sequences comprise a gap-containing position, the oligonucleotide sticks are generated by selecting as an end position a position before a gap-containing position having a gap ratio exceeding a predetermined gap ratio, and wherein the gap ratio represents a ratio between the number of gaps and the total number of bases at the gap-containing position and the total number of bases represents the sum of the numbers of existing bases and gaps,
(ii) a base exist ratio (BER) at each position of an oligonucleotide stick; wherein the BER represents a ratio between the sum of the numbers of existing bases and gaps at an alignment position corresponding to each position of an oligonucleotide stick and the total number of sequences that are aligned, and wherein the oligonucleotide stick is selected according to the number of positions each having a BER of less than a predetermined value,
(iii) a GC content; wherein a portion satisfying a predetermined GC content in an oligonucleotide stick is selected, and
(iv) amplicon region formation; wherein an amplicon region corresponding to a predetermined length in the 3′ direction from the 5′-end or in the 5′-direction from the 3′-end of an oligonucleotide stick is set, and oligonucleotide sticks included in the amplicon region are selected considering criteria regarding a stick base sum (SBS) and/or respective lengths of the oligonucleotide sticks included in the amplicon region.

28. (canceled)

29. The method according to claim 18, wherein the oligonucleotide sticks are ranked according to at least one of the following priority items:
(i) a ratio of an average base exist ratio (BER) to the number of sequence patterns of an oligonucleotide stick; the larger the ratio, the higher the priority;
(ii) an oligonucleotide stick length; the larger the length, the higher the priority; and
(iii) the number of amplicon regions in which one oligonucleotide stick is included; the larger the number, the higher the priority.

30.-31. (canceled)

32. The method according to claim 18, wherein the designable region is a designable region of oligonucleotides that permits to exhibit a maximum target coverage for the plurality of target nucleic acid sequences with at least two primer pair and/or at least two probe.

33.-34. (canceled)

35. A computer readable storage medium containing instructions to configure a processor to perform a method for determining a designable region of oligonucleotides in a plurality of target nucleic acid sequences having sequence similarity, the method comprising:
(a) selecting a start position from alignment positions of a plurality of target nucleic acid sequences;
(b) selecting as an end position a position having the minimum number of sequence patterns within the predetermined allowable number of sequence patterns from the positions located a predetermined length or more apart from the start position; wherein the number of sequence patterns is determined by a plurality of target nucleic acid sequences that are aligned,
(c) generating an oligonucleotide stick composed of a region from the start position to the end position; wherein the oligonucleotide stick comprises the number of sequence patterns and sequence pattern information determined by a plurality of target nucleic acid sequences that are aligned in the region,
(d) repeating the generation of an oligonucleotide stick by selecting at least one start position different from the start position in step (a); and
(e) determining as a designable region of oligonucleotides the regions in an alignment of the plurality of target nucleic acid sequences, which correspond to the regions of the oligonucleotide sticks.


SEQUENCE LISTING (10 sequences)

SEQ ID NO: 1 - DNA, 35 bp, Artificial
  Synthetic nucleotide Sequence 1 in Fig. 2
aaaaggggccccttttaaaagggggcccttttagc

SEQ ID NO: 2 - DNA, 34 bp, Artificial
  Synthetic nucleotide Sequence 2 in Fig. 2
aaaaggggcccattttaaaagggggcccttttgc

SEQ ID NO: 3 - DNA, 35 bp, Artificial
  Synthetic nucleotide Sequence 3 in Fig. 2
aaaaggggccccttttaaaagagggcccttttagc

SEQ ID NO: 4 - DNA, 35 bp, Artificial
  Synthetic nucleotide Sequence 4 in Fig. 2
aaaaggggccccttttaaaaggggacccttttagc

SEQ ID NO: 5 - DNA, 26 bp, Artificial
  Synthetic nucleotide Sequence 5 in Fig. 2
aaaaggggccccttttaaaaggggtc

SEQ ID NO: 6 - DNA, 34 bp, Artificial
  Synthetic nucleotide Sequence 1 in Fig. 6
aaaaggggccccttttaaaaggggccccttttaa

SEQ ID NO: 7 - DNA, 34 bp, Artificial
  Synthetic nucleotide Sequence 2 in Fig. 6
aaaaggggcccattttaaaaggggccccttttaa

SEQ ID NO: 8 - DNA, 34 bp, Artificial
  Synthetic nucleotide Sequence 3 in Fig. 6
aaaaggggccccttttaaaagaggccccttttaa

SEQ ID NO: 9 - DNA, 35 bp, Artificial
  Synthetic nucleotide Sequence 4 in Fig. 6
aaaaggggccccttttaaaagggattccttttaga

SEQ ID NO: 10 - DNA, 26 bp, Artificial
  Synthetic nucleotide Sequence 5 in Fig. 6
aaaaggggccccttttaaaagggatt